(19) United States
(12) Patent Application Publication
(10) Pub. No.: US 2004/0058851 A1
(43) Pub. Date: Mar. 25, 2004

(54) Specific autoimmune reactions against isomerised/optically inverted epitopes: application for diagnosis of autoimmune diseases

(76) Inventors: Paul Cloos, Kobenhavn (DK); Stephan Christgau, Gentofte (DK)

Correspondence Address: JONES DAY, 51 Louisiana Aveue, N.W, WASHINGTON, DC, 20001-2113, US

(21) Appl. No.: 10/075,372

(22) Filed: Feb. 15, 2002

Related U.S. Application Data
(63) Continuation of application No. PCT/EP00/07973, filed on Aug. 16, 2000.

(30) Foreign Application Priority Data
Aug. 17, 1999 (GB) 9919452.4

(51) Int. Cl. G01N 33/567; A61K 38/17

(52) U.S. Cl. 514/2; 435/7.21

(57) ABSTRACT

Antibodies and T-lymphocytes having immune reactivity with proteins isomerised at an aspartic acid, asparagine, glutamine or glutamic acid residue are found to be associated with auto-immune conditions involving auto-reactivity to IgG (rheumatoid arthritis) and myelin basic protein (multiple sclerosis). Diagnosis assays for auto-immune reactivity to isomerised protein sequences are described.

The present invention relates to methods of assay for immune system components such as auto-antibodies and auto-reactive T-cells and techniques for developing diagnostic immunoassays for initial diagnosis and monitoring of autoimmune diseases.

Autoimmune diseases comprise a complex group of conditions with the common denominator, that autologous components of the organism are recognised by the immune-system resulting in initiation of an aberrant immune-response. In order for an autoimmune reaction to occur, the normally well maintained immunological tolerance, which is maintained throughout life in healthy individuals, has to ‘break down’ (Cooke 1988) (see “References” below). The reason for this is generally difficult to assess, because the initiation of the autoimmune reactions may occur several years prior to the clinical diagnosis of the disease, and the initiating events may vary considerably in different diseases. Given the large number of potential auto-antigens in the human body it is remarkable that autoimmune diseases seem to be limited to only a few tissues and antigens. Given the localisation of the target antigen and distribution of autoimmune reactions in the organism, autoimmune diseases may be classified as either organ specific or non-organic specific (systemic). In either case the immuno-reactions may involve both the humoral (i.e. antibody synthesising) and the cellular part of the immune system (Cooke 1988).

The present invention relates to techniques for characterising immune system components such as autoantibodies and auto-reactive T-cells or B-cells and molecules which are interactive therewith such as auto-antigens, for detection and quantification of such immune system components and auto-antigens. In an example of the application of the techniques and uses of the invention, autoimmune phenomena associated with the systemic autoimmune disorder rheumatoid arthritis (RA) or multiple sclerosis (MS) are described. However this is merely meant as an illustration of the invention, and by no means intended to limit the scope of the invention to only RA or MS.

Hypothesis Underlying the Invention

The present invention is based on the hypothesis that isomerisation and optical inversion of susceptible residues in proteins may be important for the generation of an autoimmune response in autoimmune diseases. Aspartic acid and asparagine (Asx) and glutamic acid and glutamine (Glx) residues will in some susceptible proteins undergo a spontaneous rearrangement where the normal peptide bond between the Asx or Glx residue and the adjacent residue is transferred from the normal α-carboxyl group to the β-carboxyl group (γ-carboxyl group for the Glx residues) of the side chain (Clarke 1987). The isomerisation reaction proceeds via a succinimide intermediate, which upon spontaneous hydrolysis may result in one of four forms: the normally occurring αL, the isoform βL, or the two optically inverted forms αD and βD as outlined in the following reaction scheme for aspartic acid:

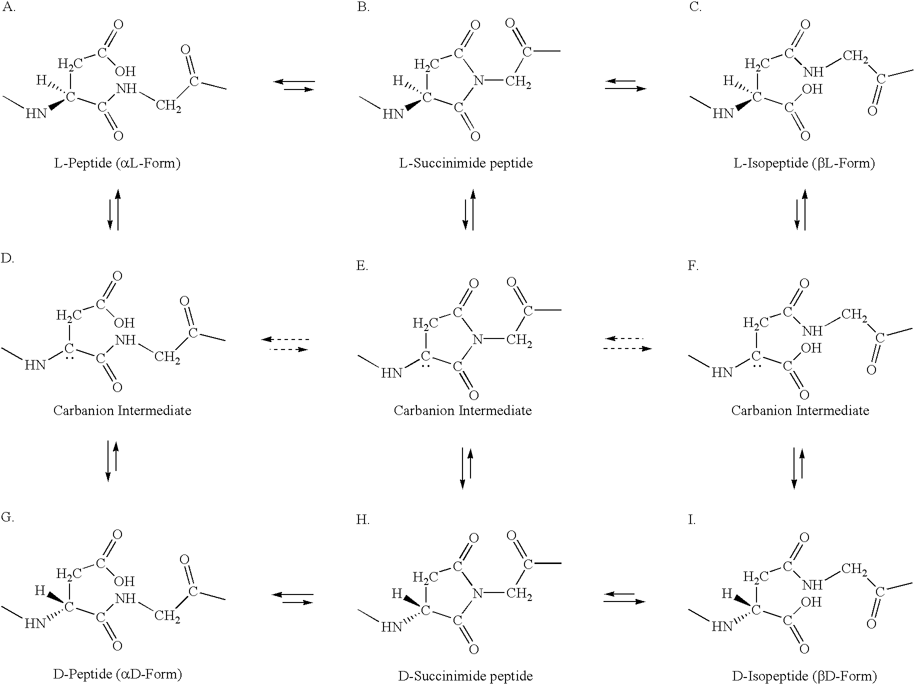

The attack by the peptide backbone nitrogen on the side chain carbonyl group of an adjacent aspartyl residue can result in the formation of a succinimide ring, (A→B). The succinimide ring is prone to hydrolysis and optical inversion yielding peptides and isopeptides in both the D and L configurations. Optical inversion proceeds through a carbanion intermediate (D, E and F) either through direct proton abstraction (A⇄D⇄G or C⇄F⇄I) or via the succinimide pathway (D⇄E⇄H). Throughout the figure the peptide backbone is shown as a bold line. The figure depicts the isomerisation/optical inversion reaction occurring at an Asp-Gly sequence but the reaction can occur at any susceptible Asx or Glx containing epitope.

However, in order for cyclic imide formation (and isomerisation/optical inversion) to occur, the three dimensional structure surrounding the Asx or Glx residues must have an optimal conformation and sufficient flexibility (Clarke 1987).

Studies indicate that optical inversion of Asx residues in peptides and proteins primarily proceeds through the succinimide pathway (B⇄E⇄H) (Geiger and Clarke 1987, Radkiewics et al 1996). However, other pathways such as direct proton abstraction or imino-δ-lactone formation may also contribute to optical inversion (Radkiewics et al 1996). These pathways are however assumed to be of less importance (Geiger and Clarke 1987, Radkiewics et al 1996).

The introduction of such structural changes in a protein or peptide has profound effects on its function, stability and physical and chemical properties. The invention describes the role of such structural changes for the immunogenicity of the molecules.

Introduction of an isomerised and/or optically inverted residue in a protein results in the novel βL, αD and βD forms which play an important role for the ability of the protein to elicit an immune response. Especially in autoimmune diseases, where autologous components of the patients own tissues or organs suddenly become targets of the immune system as antigens, isomerisation and/or optical inversion play an important role.

The isomerisation and/or optical inversion which occurs spontaneously at a very low rate can introduce a novel epitope in the molecule which is unlikely to be subject of immunological tolerance. Such a novel epitope can be recognised by the antigen presenting cells of the immune system and thus elicit an immune response.

It has been proposed that the effect of ‘protein fatigue’ related to Asx damage on protein properties including immunogeneity may be worth investigating (Galletti et al 1995).

It has been reported that immunological properties of short peptides may be influenced by the optical inversion of aspartyl residues (Benkirane et al 1993).

It has further been reported that in vitro de-amidated serum albumin has altered antigenic properties, making it immunogenic in the body and that a similar process in vivo might play a role in the development of autoimmune processes in the ageing body (Lukash et al, 1987).

Deamidation of asparagine may be a consequence of isomerisation of the peptide bond but there are a number of other processes of deamidation (Mor et al, 1992). Deamidation as such does not cause a structural change in the backbone of the protein of the kind caused by isomerisation/optical inversion via a succinimide intermediate in the manner shown in the reaction scheme shown above. Deamidation may for instance be the result of the action of an enzyme specific for removal of an amine —NH₂ group of the amide, which does not alter the peptide bond or involve any change in optical activity.

It has been reported, that the immune response to peptides containing D-amino acids is different from the response to corresponding peptides composed exclusively of L-amino acids (Sela & Zisman 1997, Maillère et al 1995, Todome et al 1992, Sela & Fuchs 1965). Todome et al (1992) demonstrated that a bacterial protein fragment containing D-alanine residues is able to raise an immune response in humans. Maillère et al (1995) showed that substitutions of normally occurring L-amino acids in a T-cell epitope, derived from a snake venom, alter its binding and reactivity with the T-cell receptor. Sela and Fuchs (in a conference proceeding from a meeting held Prague in 1964) describe that inclusion of a D-amino acid in an epitope/antigen may increase (or decrease) its antigencity, as assessed from experimental work performed with synthetic oligo-peptides containing D-tyrosine. These and similar observations are further discussed in the reviews by Mor et al (1992) and Sela and Zisman (1997).

None of these reports discuss the possibility that auto-antigens or auto-antigenic epitopes may contain D-amino acids; or that the occurrence of such a D-amino acid may induce an auto-immune response. Furthermore, none of the studies cited above involves spontaneous optical inversion via the succinimide pathway as described in this patent, but rather they describe work carried out with synthetic peptides made with D-amino acids other than *Glx and *Asx.

Rheumatoid Arthritis

Rheumatoid arthritis (RA) is a severe chronic and progressive disease affecting approximately 1% of the population in both the industrialised and the developing world. Although both environmental, genetic and developmental factors have been implicated in the aetiology of RA, it is now well established that RA is an autoimmune disease (Williams 1996).

The major clinical manifestation of RA is an abnormal and degraded cartilage and synovial tissue, resulting in a severe reduction of the lubricating function of the joints, and consequently motility problems in the RA patients. The affected joints show infiltration (synovitis) containing polymorphonuclear neutrophils, macrophages, T-cells and other cells of the immune-system. These cells take part in an active immunological process, where the action of these cells and their secreted products are mediators of joint destruction (Munthe & Natvig 1972; Harris 1993). In turn, the active synovitis results in the outgrowth of new capillaries (angiogenesis) and synovial lining cells into the joint, further hampering its normal function (Munthe & Natvig 1972).

The most characteristic serological feature of RA is the presence of circulating antibodies directed against autologous IgG (Bernstein 1990). These anti IgG auto-antibodies are referred to as rheumatoid factors (RFs). RFs can be both IgM, IgG, IgA and IgE, but IgM and IgG RFs appear to have the major clinical significance and prevalence among RA patients (Jonsson & Valdimarsson 1993). The finding that many immunoglobulin classes are involved in the RF response strongly suggests, that RF formation is antigen driven and T-cell dependant and not merely the result of monoclonal proliferation or a general stimulation of the immune-system (Harris 1993; Bernstein 1990).

RFs are not specific to RA, they are also found in the sera of a variable portion of patients with acute inflammation diseases, autoimmune diseases and of some apparently normal individuals (Chen et al 1987, Carson et al 1993, Bernstein 1990). The formation of self-associating RF complexes locally in synovial tissues is seen only in RA and other systemic autoimmune diseases such as Sjögrens syndrome, systemic lupus erythematosus and sclerodera (Natvig & Munthe 1975, Winchester 1975), suggesting that some abnormal factors or immunological responses accelerate the aggregation of IgG-RF complexes in these diseases.

The initiating, causative factor(s) for RA has not been identified, and as is the case with most autoimmune diseases such studies are difficult to carry out, because the autoimmune attack may be initiated several years prior to the clinical manifestations of the disease. It is well established that higher susceptibility to RA is associated with certain MHC gene alleles, namely the Dw4 and Dw14 genes of the DR-1 locus (Nepom 1990).

The role of RF in the initiation and pathogenesis of the disease is unknown, and the question whether RF is a central event in the disease or merely arises as a secondary phenomena in RA remains unanswered. Initiation of RF formation in RA could be the result of conformational changes in the Fc regions of IgG (Johnson et al 1975).

The observation by Parekh et al. (1985) that IgG isolated from patients with RA showed defective galactosylation of oligo-saccharides in the Fc fragment caused great excitement, but further research into the clinical implications of the IgG-glycosylation status has given conflicting results (Parekh et al. 1988, Tomana et al. 1988). Rather, defective galactosylation of IgG may be a general risk factor for developing autoimmune diseases (Harris 1993; Pilkington et al. 1995).

Immuno-histochemical studies of joints from RA patients have revealed considerable numbers of IgG containing plasma cells manifesting RF activity in rheumatoid synovia (Munthe & Natvig 1972), and it has been proposed that RFs react with autologous IgG forming large self-aggregating complexes which in turn may be phagocytosed and result in the subsequent release of lysosomal enzymes. Several observations support such a mechanism of immunologically triggered tissue damage in RA (Williams 1996; Carson 1993).

The heterologous nature of RFs with several immunoglobulin types, and multiple epitopes on the IgG molecule involved, has also hampered accurate assessment of their role in the disease (Kalsi & Isenberg 1993). RA associated RFs may be different from RF's found in other situations and they are apparently directed against epitopes in the C_{H}2 and C_{H}3 domain of the Fc region of IgG (Bonagura et al 1993).

IgG contains a number of asparagine and aspartic acid (Asx) residues which may theoretically be subject to cyclic imide formation (isomerisation/optical inversion). The three dimensional structure of IgG is well known, and was included in a theoretical study of potential sites for Asx isomerisation in human proteins (Clarke 1987). Assuming standard bond lengths and geometry, Clarke calculated the distance from the backbone nitrogen atom to the side chain gamma carbonyl carbon of Asx or Glx residues in a variety of proteins including human IgG, based on the dihedral angles phi, psi, chi and chi₂. These theoretical considerations indicated that Asn-384 in the human IgG FC_{H}3 region requires only minimal conformational changes for imide formation. It may therefore be anticipated that this site tends to isomerise (Clarke (1987). In addition, studies by Svasti and Milstein (1972) have shown that mouse IgG is isomerised at Asn-Gly sequences in the Fc fragment (Svasti & Milstein 1972). The region around Asn-384 is surface exposed and may be especially sensitive to environmental influences promoting imide formation.

Autoimmune Reactions and Multiple Sclerosis

Multiple sclerosis (MS) is an inflammatory disease of the central nervous system (CNS) white matter that results in areas of demyelination with disruption of neurologic function. The pathogenesis of MS remains to be elucidated but is believed to result from autoimmune mechanisms leading to myelin destruction.

The initiating, causative factor(s) for MS has not been identified, and as is the case with most autoimmune diseases such studies are difficult to carry out, because the auto-immune attack may be initiated several years prior to the clinical manifestations of the disease. Several events must occur before the MS disease process reaches a pathological level. Among these events are breakdown of the normal immunological tolerance to myelin proteins and defects in the normally well maintained blood brain barrier that normally prevents contacts between components of the CNS and the immune-system (de Vries et al 1997).

The role of auto-antigens in the initiation and pathogenesis of the disease is unknown, and the question whether the formation of auto-antigens is a central event in the disease or merely arises as a side phenomenon in MS remains unanswered. Initiation of auto-antigen formation in MS could be the result of conformational changes of myelin proteins.

Several myelin proteins have been implicated as targets of auto-antibodies and auto-reactive T cells, including Myelin Basic Protein (MBP) and Myelin Oligodendrocyte Glycoprotein (MOG) and αB-crystallin (Martin 1997, Bettadapura et al. 1998, Van Noort et al 1998).

MBP is a target of auto-antibodies and auto-reactive T-cells in MS. The three dimensional structure of MBP is well known (Beniac 1997) and the molecule contains a number of asparagine and aspartate residues which may theoretically be subject to cyclic imide formation (isomerisation/optical inversion). It is likely that isomerisation/optical inversion affects antigenicity of the protein.

MOG is a trans-membrane glycoprotein localised to the external surface of myelin sheaths (Linington et al. 1984). Due to its restricted localisation on the outer surface of the myelin sheaths, MOG, provides an ideal primary target antigen for autoimmune attacks in MS particularly because the presence of anti-MOG antibodies within the CNS causes extensive demyelination, both in vivo and in vitro (Adelman et al 1995). MOG is the only myelin auto-antigen so far described that can initiate both a demyelinating antibody in EAE models and also contains an encephalitogenic T-cell epitope (Linington et al. 1993). In addition the presence of anti-MOG antibodies has been demonstrated in blood and CSF of MS patients (Sun et al. 1991). Moreover Kerlero de Rosbo and colleagues have demonstrated a predominant T-cell response to MOG in a population of MS patients (Kerlero de Rosbo et al. 1993). MOG contains only one potential site of optical inversion/isomerisation. This site comprises residues 54-55 of MOG and is located at the surface exposed part of the molecule. In addition, this site is a part of the sequence MOG₃₅₋₅₅ which has been shown to be highly encephalitogenic and a strong (the strongest) inducer of S and T-cell responses (Ichikawa et al.1996).

We therefore propose that potential isomerisation of MBP, αβ-crystallin or MOG at susceptible sites may play a role in MS pathogenesis: Isomerisation/optical inversion may be directly involved in the initial phases of MS by providing novel immunogenic epitopes, which will become targets for the humoral and cellular immune-system.

Other Autoimmune Diseases

The theory underlying the present invention (i.e. that isomerisation/optical inversion of self-proteins create new untolerised epitopes causing an autoimmune response) may likewise be applied to other antigen-driven autoimmune diseases, among these:

Insulin-Dependent Diabetes Mellitus (IDDM)

Pancreatic β-cells in islets of Langerhans can be destroyed as a consequence of autoimmune reactions resulting in insulin-dependent diabetes mellitus (IDDM). The destruction proceeds over a long period of time before the onset of clinical symptoms (Gorsuch et al. 1981). A number of self-proteins have been identified as auto antigens in IDDM.

The neuroendocrine enzyme glutamic acid decarboxylase (GAD) is a major auto-antigen in IDDM (Baekkeskov et al. 1990). Two GAD isoforms exist, GAD65 and GAD67, which differ mostly in the first 100 amino acid of the N-terminus. IDDM sera are predominantly reactive with GAD65, but auto-epitopes are primarily localised to regions of GAD65 highly homologous with GAD67.

The two closely related proteins IA2 and IA2beta which belong to the family of membrane spanning tyrosine phosphatases, (Bonifacio et al. 1995, Lu et al. 1996) have likewise been shown to be auto-antigens in IDDM. IDDM patients frequently display auto-antibodies against these proteins (Li et al. 1997).

Glima38 is a 38 kDa Islet cell membrane glycoprotein which has also been shown as an auto-antigen in IDDM (Baekkeskov et al. 1982, Aanstoot et al. 1996).

In addition insulin auto-antibodies (IAA) are detected in at least half of all newly diagnosed IDDM patients (Palmer et al 1983), however the predictive power of IAA is poor (Dean 1986).

Myasthenia Gravis (MG)

Myasthenia gravis (MG) is a organ specific autoimmune disease targeting the skeletal muscle acetylcholine receptor (AChR) (Berrih-Aknin 1995). Thus, most MG-patients have auto-antibodies directed against AChR that interfere with neuromuscular transmission. Although AChR is present in the thymus, tolerence to this protein is lacking in MG patients. One explanation for this observations may be that MG patients mount an immunological response towards “altered” (isomerised or optically inverted) AChR which constitutes a new epitope and consequently is not tolerised. Several T-cell epitopes on AChR have been described (Zisman et al. 1996, Yoshikawa et al. 1997, Atassi & Oshima 1997) among these AChR¹²⁹⁻¹⁴⁵ containing several Asx and Glx residues which potentially could be prone to isomerisation/optical inversion. Recently, another possible auto-antigen in this disease has been identified: Gravin, a 250 kDa kinase scaffold protein (Nauert et al. 1997). Correspondingly Asx and Glx residues in this protein may be prone to isomerisation/optical inversion inducing an autoimmune response.

Celiac Disease (ClD)

Celiac disease (ClD) is characterised by IgA auto-antibodies to the endomysium and T-cell mediated hyper-sensitivity to gluten in food. Gliadin has been demonstrated to be the immunogenic part of gluten which reacts with T-cell clones from ClD patients. The intestinal inflammation in ClD is precipitated by exposure to wheat gliadin in the diet and is associated with increased mucosal activity of the enzyme tissue transglutaminase (TGase). This enzyme (TGase) has been identified as an auto-antigen in this condition (Dieterich et al. 1997). Thus 98% of patients have elevated IgA titres against (TGase), whereas 95% of healthy controls are negative (Dieterich et al. 1998).

Chagas' Disease (CD)

Infection with the protozoan parasite Trypanosoma cruzi often results in chronic heart and gut-associated autoimmune disease called Chagas' disease (CD). The chronic disease is characterised by rich inflammatory infiltrate in myocardial and nervous tissues. A number of self-proteins have been identified as auto antigens in CD among these cardiac myosin, (Abel et al. 1997), muscarinic acetylcholine receptor (mAChR) (Goin et al. 1997), and small nuclear ribonucleoprotein (UsnRNP) (Bach-Elias et al. 1998).

Psoriasis (Ps)

Psoriasis (Ps) is a proliferative chronic disease of the epidermis that appears to be of autoimmune nature. The typical clinical manifestation of the disease is inflamed swollen skin lesions covered with a silvery white scale. However the disease comes in many different variations and degrees of severity. Five to ten percent of Ps patients develop psoriatic arthritis which causes inflammation and erosion of joints. The pathogenesis of the disease is still open to debate, but the autoimmune nature of the disease is substantiated by the well-known success of immuno-suppressive treatments and IL-2 toxin (a drug which selectively blocks the growth of activated T-cells) (Gottlieb et al. 1995). Several studies likewise suggest the involvement of T-cell pathogenesis in the disease (Schon et al. 1997). A putative 200 kDa lamina lucida Ps-autoantigen has recently been identified (Chen et al. 1996).

Crohn's Disease (CrD)

Crohn's disease (CrD) is a chronic inflammatory disease of the intestines. It is most often located to the small and large intestines where it causes ulcerations, but CrD can affect the digestive system anywhere. The cause of CrD is unknown at present but the disease appears to be autoimmune in nature however at present no auto-antigens or T-cell epitopes have been identified.

### SUMMARY OF THE INVENTION

The present invention now provides a method of assay comprising subjecting a sample to a quantitative or qualitative determination of the presence in the sample of (a) an auto-reactive immune system component specifically recognising an epitope containing an isomerised peptide linkage and/or an optically inverted amino acid, and/or (b) an auto-antigen or a fragment thereof containing a said epitope and/or (c) a non-self antigen or fragment thereof which contains a said epitope and is capable of inducing an autoimmune response.

The isomerisation may be at an aspartic acid or asparagine amino acid residue or a glutamic acid or glutamine amino acid residue.

Said immune system component may be a cellular immune system component, e.g. a T-lymphocyte.

Alternatively, said immune system component may be a humoral immune system component such as an antibody. The antibody may be of any of the known antibody types, especially IgG.

Said epitope may comprise an amino acid sequence of essentially any protein, but in relation to some autoimmune conditions may be an isomerised or optically inverted IgG, MOG, MBP or αβ-crystallin. In relation to other autoimmune conditions, the epitope may form part of a protein attacked during the progression of the disease.

The detection of said auto-antibodies is preferably indicative of an autoimmune disease, for instance rheumatoid arthritis, multiple sclerosis, insulin dependent diabetes mellitus, myasthenia gravis, celiac disease, Chagas' disease, psoriasis, or Crohn's disease.

Said immune system component may be an auto-antibody directed against an epitope comprising the or an amino acid *Asx contained in any one of the sequences:

Trp-Leu-*Asx-Gly-Lys-Glu-Tyr
Trp-Glu-Ser-*Asx-Gly
His-Phe-Phe-Lys-*Asx-Ile-Val-Thr-Pro
Pro-Ser-*-Asx-Glu-Gly-Lys-Gly-Arg
Ala-Leu-Gly-Ile-Gly-Thr-*-Asx-Ser-Val-I1e
Trp-Ser-Phe-Gly-Ser-Glu-*Asx-Gly-Ser-Gly-*Asx-Ser-
Glu-Asn
Met-Glu-Val-Gly-Trp-Tyr-Arg-Pro-Pro-Phe-Ser-Arg-
Val-Val-His-Leu-Tyr-Arg-*-Asx-Asn-Gly-Lys
Val-Val-His-Phe-Phe-Lys-*Asx-Ile-Val-Thr-Pro
Ala-Gly-Trp-Leu*Asx-Gly-Ser-Val-Arg or
Gly-Arg-Val-Arg-Val-*Asx-Ser-Ala-Tyr.

where *Asx is αD Asn or Asp, or is βD, or βL Asp formed by isomerisation/optical inversion of Asp or Asn residues in the original sequence.

Said immune system component may be an auto-antibody directed against an epitope comprising the amino acid Glx* contained in any one of the sequences:

Pro-Ser-*Glx-Gly-Lys-Gly-Arg
Phe-Ser-Trp-Gly-Ala-*Glx-Gly-Arg or
Asp-Ala-*Glx-Gly-Thr-Leu-Ser-Lys

where *Glx is αD, Glu or Gln, or is γL or γD Glu formed by isomerisation/optical inversion of Glu or Gln residues in the original sequence.

The epitope in question may be a T-cell epitope or a B-cell epitope.

The invention includes a method for the detection of an auto-antigen or fragment comprising detecting the reactivity of said auto-antigen or fragment with an immunological binding partner specific for the presence in said auto-antigen of an isomerised peptide linkage or a optically inverted amino acid.

Preferably, said auto-antigens are associated with an autoimmune disease.

The invention includes methods in which one detects a non-self antigen which produces an immunological response which is cross-reactive with a self epitope containing an isomerised or optically inverted amino acid. Thus, exposure to a non-self origin mimic of a self-protein sequence may induce an immune response which then becomes directed against the self-protein in a disease producing manner. Alternatively, the non-self antigen may produce a response such as an inflammatory response with immune system involvement leading to a breakdown in self tolerance and hence to the production of an autoimmune response to other epitopes not present on the triggering non-self antigen (epitope spreading). According to this aspect of the invention, there is provided a method for the detection of a non-self antigen or fragment thereof which produces an autoimmune condition comprising detecting the reactivity between said antigen or a fragment thereof and an immunological binding partner specific for the presence in said antigen of an isomerised peptide linkage or an optically inverted amino acid.

Such methods may provide information as to the amount of said auto-reactive immune system component or auto-antigen or fragment detected or may be purely qualitative.

These putative immune responses in response to isomerisation may be of primary importance for the disease, i.e. they may be the initiating or causative factor. Alternatively they may be of secondary importance developing as a result of the other immune and cellular processes occurring as a result of the disease. In either case, both the humoral and cellular components of the immune system may be involved. Thus, the aim of the present invention is to develop diagnostic agents able to detect or quantify the presence of specific components of the immune system (such as antibodies) directed against isomerised (and/or optically inverted) target epitopes of specific antigens, thus facilitating diagnosis and monitoring the disease. Where the immune response in question is secondary rather than causative in the context of the disease, the altered immune response may still be of diagnostic relevance.

As further explanation of a specific embodiment of the invention, we describe below the involvement of isomerisation of specific IgG sequences in the pathogenesis of RA. However, this is not meant to limit the scope of the invention to exclude use for other autoimmune diseases, where isomerisation or optical inversion of key antigens occurs and results in similar reactions to those described below.

As discussed above Asn-384 of IgG is one potential site for isomerisation (Clarke 1987). However, this residue is not the only surface exposed asparagine residue in the Fc region of IgG, which may be subject to isomerisation. In example 1 below direct evidence is shown that Asn-315 is subject to isomerisation and this residue is also surface exposed, localised in the Fc_{H}2 region (Bonagura et al 1993).

Having regard to the findings of Tomiyama et al (1994) that the aggregation properties of beta-amyloid are affected by the optical inversion of Asx residues depending on their position, it is not inconceivable that isomerisation/optical inversion of IgG changes the solubility and hydrophilicity of IgG to induce IgG auto-aggregation. Furthermore, according to our findings isomerisation/optical inversion almost certainly affects antigenicity of the protein. Individuals with reduced clearance of IgG from the bloodstream and/or elevated IgG concentrations may be more prone to this. Additional factors may aggravate this situation, such as other serum proteins or allelic variations of IgG genes decreasing their solubility or increasing their ability to aggregate. Environmental factors may also influence this situation by modulating the function of the immune system.

Therefore, potential isomerisation/optical inversion of IgG at susceptible sites may play a role in autoimmune disease pathogenesis in two ways:

A) First, isomerisation/optical inversion may be directly involved in the initial phases of RA by providing novel immunogenic epitopes, which will become targets for the humoral immune-system. Specific antibodies recognising the isomerised or optically inverted autologous IgG will thus arise and they could play a primary role in the disease by generating large insoluble immuno-complexes which aggregate in synovial tissue of joints where they initiate an inflammatory response (Inman & Day 1981). Furthermore the cellular component of the immune system may also become targeted towards such a novel epitope and mediate some of the destruction in the synovial tissue characterising RA.

B) Alternatively, the aggregation of IgG in RA may lower IgG clearance and therefore the aggregated IgG may isomerise/optically invert as a function of retention time. Especially the synovial fluid with a low clearance rate may be a likely place for this process to take place. Thus, in this scenario, the isomerisation/optical inversion arises as a sign of IgG aggregation associated with the RA process, but it may eventually lead to the formation of Iso-IgG specific auto-antibodies as described above. Again, the cellular component of the immune-system may become involved at this stage.

The two hypotheses outlined above are not mutually exclusive, and in both cases auto-antibodies recognising isomerised or optically inverted Asx or Glx residues would be specific markers of the disease. Among the observations that support the views that isomerisation of susceptible asparagine or aspartic acid residues is involved in the autoimmune reactions characterised by the occurrence of rheumatoid factors are the following: RFs recognise epitopes on C_{H}2 and C_{H}3, where all potential isomerisation sites are situated (Johnson and Page Faulk 1976; Nardella et al. 1981). Several lines of evidence indicate that aberrant (not normal) IgG is present in RA (Rawson et al. 1969; Watkins et al. 1972; Johnson et al. 1974). Finally, the steric conformation and amino acid sequence around Asn₃₁₅ in the C_{H}2 and Asn₃₁₄ in the C_{H}3 region of IgG1, 2 & 4 are almost optimal for imide-bond formation (Clarke 1987), and the potential epitopes are surface exposed.

Glant et al have reported that cartilage aggrecans contain epitopes to which an autoimmune response can be generated in mice. Aggrecan is a proteoglycan constituent of cartilage in which we have identified the potential isomerisation/optical inversion site contained in the amino acid sequence Gly-Arg-Val-Arg-Val-Asu-Ser-Ala-Tyr in the G-1 domain of aggrecan. Autoimmune responses to an isomerised and/or optically inverted epitope defined in this sequence may be targeted in this invention.

The cartilage link protein (CLP) which is associated in cartilage with aggrecan and hyaluronan has also been shown to have relevance in that autoimmunity against this protein can induce RA in an animal model of the disease (Zhang et al 1998). We have identified the sequence Ala-Gly-Trp-Leu-Ala-Asp-Gly-Ser-Val-Arg as a potential isomerisation and/or optical inversion site which may be involved in autoimmunity.

By analogy, autoimmune responses against other key auto-antigens such as MBP or MOG in MS or more generally isomerized or optically inverted antigen in an autoimmune disease may play a similar role in the pathogenesis of the disease in question.

In a further aspect of the invention, other autoimmune diseases may be characterised by susceptibility of key antigens to isomerise or to occur in optically inverted forms, and thus generate immune responses of primary or secondary importance for the disease. Thus, the invention is not limited to diagnostic agents for RA or MS, but applies generally also for other autoimmune diseases.

For the purposes of carrying out the invention, isomerisation/optical inversion of key epitopes in target auto-antigens of autoimmune diseases may be identified by one or more of the procedures listed below, if the target antigen is known.

If the three dimensional structure of the target antigen is known, potential isomerisation/optical inversion sites can be identified (e.g. Asx-Gly sequences). Their theoretical propensity for isomerisation/optical inversion can be assessed based on calculation of the dihedral angles phi, psi, chi and chi, and the flexibility of the amino acid side chain containing the β-carboxyl group (Clarke 1987). Furthermore, it can be assessed whether the potential altered residue is surface exposed and thus accessible for autoantibodies. An important parameter is the half life of the protein, because only proteins with a relatively extended half life (say more than 10 days) can be expected to undergo isomerisation and/or optical inversion to a significant extent.

It will be appreciated that the invention can usefully be practised to detect immune system components having specificity for *Asx or *Glx containing epitopes even if the altered protein in question is not causative of the disease in question and the production of the auto reactive immune system component is symptomatic rather than causative. Recognition of the presence of the auto reactive component may in such cases have valuable diagnostic significance as regards initial diagnosis and monitoring of therapy.

The invention includes a method for locating an epitope or epitopes in an auto-antigen comprising using L-iso-aspartyl (D-aspartyl) methyl-transferase (IAMT)-EC 2.1.1.77 and a source of labelled methyl groups to introduce said labelled methyl groups at one or more isomerised or D-form aspartic acids in said auto-agtigen, and determining at least one location in said auto-antigen at which said labelled methyl groups are thus introduced, establishing the amino acid sequence of said auto-antigen in a region encompassing a said location and testing a peptide of said amino acid sequence incorporating at said location said isomerised or optically inverted amino acid for immuno-reactivity with an auto-reactive immune system component, e.g. with auto-antibodies.

Thus the target antigen of interest (e.g. glutamic acid decarboxylase in type I diabetes, Myelin basic protein or MOG in multiple sclerosis, or IgG in Rheumatoid arthritis), can be analysed by the enzyme IAMT. This enzyme recognises αD and βL Asx (but not βD Asp and not altered Glx) i.e. certain isomerised or optically inverted aspartic acid and asparagine residues, and methylates the α-carboxyl group. By employing a radioactively labelled methyl-donor, isomerised proteins or peptides incubated with this enzyme will be radioactively labelled, and labelling of the protein can be detected by measuring the incorporated radioactivity.

By fragmenting the antigen of interest either by chemical or proteolytic hydrolysis, and purifying the generated fragments by known chromatographic methods, followed by analysis of the fragments by the IAMT assay the position of the isomerised site can be identified. Fragments identified as containing an isomerised sequence by the IAMT assay can be subjected to amino acid sequencing and amino acid analysis in order to pinpoint their exact localisation in the target antigen.

Relevant isomerised sequences that may be identified by the use of IAMT or other methods described herein include:

from IgG:
(RA)
Asn-315: Trp-Leu-*Asx-Gly-Lys-Glu-Tyr, His-Gln-Asp-Trp-Leu-
*Asx-Gly, His-Gln-Asp-Trp-Leu-*Asx-Gly-Lys-Glu-Tyr.
Asn-384: Trp-Glu-Ser-*Asx-Gly-Gln-Pro-Glu, Val-Glu-Trp-Glu-
Ser-*Asx-Gly, Val-Glu-Trp-Glu-Ser-*Asx-Gly-Gln-Pro-Glu.
from MBP:
(MS)
Asn-92: His-Phe-Phe-Lys-*Asx-Ile-Val-Thr-Pro
Gln-103: Pro-Ser-*Glx-Gly-Lys-Gly-Arg
Gln-119: Phe-Ser-Trp-Gly-Ala-*Glx-Gly-Arg
Gln-143: Asp-Ala-*Glx-Gly-Thr-Leu-Ser-Lys
from MOG
(MS)
Asn-53: Met-Glu-Val-Gly-Trp-Tyr-Arg-Pro-Pro-Phe-Ser-Arg-
Val-Val-His-Leu-Tyr-Arg-*Asx-Gly-Lys
from GAD₆₅:
(type I diabetes)
Asp-297: Ala-Leu-Gly-Ile-Gly-Thr-*Asx-Ser-Val-Ile
Asp-15 & 19: Trp-Ser-Phe-Gly-Ser-Glu-*Asx-Gly-Ser-Gly-*Asx-
Ser-Glu-Asn

Where:

*Asx is αD Asp or Asn, or is βD, or βL Asp formed by optical inversion/isomerisation of Asp or Asn, and

*Glx is αD Glu or Gln, or is γD, or γL Glu formed by optical inversion/isomerisation of Glu or Gln

Included within the scope of the invention are peptides containing an epitope which is also present in any one of these amino acid sequences. Also included are other peptides containing isomerised or optically inverted amino acid containing epitopes located by the use of L-iso-aspartyl(D-aspartyl)methyl-transferase (IAMT).

If the auto-antigen has not been identified in the autoimmune disease of interest, the target tissue or organ for the autoimmune destruction may be analysed. Solubilisation and proteolytic degradation of the tissue as described above, may be followed by purification of the generated peptides by chromatographic or other techniques, and use of the IAMT assay for identification of isomerised/optically inverted fragments. These may then be identified by amino acid sequencing, amino acid analysis, mass spectrometry and other relevant methods.

Detection of Auto-Antibodies to Isomerized or Optically Inverted Epitopes

Auto-antibodies from human patients or animal subjects recognising isomerised/optically inverted peptide sequences in major epitopes of key auto-antigens may be detected by assays as described below.

Generally, a wide range of known immunoassay formats and procedures may be employed, including ELISA, RIA, hetero-geneous and homogeneous assay procedures. By way of example, synthetic isomerised or optically inverted peptides, or proteolytically generated fragments of an authentic antigen containing the epitope of interest may be coated to the solid phase of a microtitre plate (MTP), either conjugated to a carrier protein (e.g. thyroglobulin or serum albumin) or by being biotinylated and thus being able to bind to a streptavidin coated MTP surface. Reactive auto-antibodies may then be identified by adding serum samples suitably diluted in assay buffer to the wells of the MTP, where they will bind to the immobilised epitope containing material. The amount of bound antibody can be quantified by the use of a secondary enzyme conjugated, anti-human antibody followed by a chromogenic enzyme substrate. Care must be taken in this assay system to minimise non-specific reactions due to absorption of IgG or other serum components to the MTP surface.

Alternatively, antibodies may be raised against the epitope in question, and these antibodies may be immobilised to an MTP surface. A synthetic peptide containing the isomerised epitope in question, or a proteolytically generated fragment of the authentic antigen containing the target epitope, may then coupled to either an enzyme such as peroxidase or alkaline phosphatase, or it may be labelled with a ligand such as biotin or digoxigenin. This reagent is then added in a suitable dilution to the wells together with a serum sample. Auto-antibodies in the serum sample reactive with the target epitope will block the epitopes binding to the antibodies coated to the MTP surface and thus result in a decrease in the signal which can be generated by a subsequent addition of a chromogenic enzyme substrate, or a streptavidin conjugated detection agent. The signal can be quantified and used for assessment of the amount of auto-antigens in the investigated sample.

Another competitive assay format employing non-human antibodies raised against the epitope in question as described above may be performed using MTP plates coated with synthetic or authentic peptide or peptide fragments containing the epitope in question. The peptide may be coated either directly to the MTP surface or conjugated to a carrier protein (e.g. thyroglobulin or serum albumin), or it may be biotinylated and thus be made able to bind to a streptavidin coated surface. Human serum samples appropriately diluted in assay buffer are incubated on the MTP followed by or simultaneously with antibodies raised against the epitope in question. Serum samples containing auto-antibodies reactive with the epitope in question will react with the epitopes provided on the MTP surface and thus displace the binding of the other antibodies. By using an enzyme labelled secondary antibody specific for the non-human antibodies raised against the epitope in question the amount of bound human antibodies can be quantified after incubation with a chromogenic enzyme substrate. The amount of dye will be inversely proportional to the amount of bound human auto-antibody.

A homogeneous assay format may be performed by incubating a suitably diluted human serum sample with a biotinylated peptide containing the epitope in question, and streptavidin covalently labelled with an appropriate enzyme or with radioactive molecules such as ¹²⁵I. Auto-antibodies present in human serum sample will bind to the target epitope on the streptavidin molecule, and they can then be precipitated with either Protein A Sepharose, or another precipitation agent or solid phase specific for human IgG. The amount of bound antibody can then be quantified by use of a chromogenic enzyme substrate in the case of enzyme labelled streptavidin, or by scintillation counting in the case of streptavidin labelled with a radio-isotope.

Detection of Cellular Immuno-Reactivity to Isomerized/Optically Inverted Auto-Antigens.

Studies in vitro of the autoimmune responses from newly diagnosed patients with autoimmune diseases are highly desirable in order to aid in diagnosing the disease, assisting in therapy selection and monitoring and calculation of prognosis for the individual patient. Determination of the interactions of molecular components of the immune-system involved in the autoimmune response is necessary to characterise the disease and assess the importance of antigens and cellular responses for the development of the disease. The previous paragraphs describes the analysis of the humoral reactivity against target epitopes in either βL αD or βD form for Asx residues (as well as γL or γD forms for Glx residues). However, the cellular immune response against altered autologous proteins may be of equal/greater importance for the monitoring of the disease. The cellular compartment of the immune system is involved in most autoimmune diseases, including RA and MS where T-cells have been mentioned as primary mediators of tissue destruction. Determination of the targets for the cellular compartment of the immune-system can be essential in order to determine whether the immune responses are of primary or secondary importance.

Detection of T-cell mediated autoimmunity can be performed by several methods, such as: T-cell proliferation assays, ELISPOT assays, limited dilution assays, or ⁵¹Cr-release assays (for a general overview of the methods, please see C. A. Janeway & P. Travers (1997). Below is given a short outline of some of these methods, which may be used to study cellular immuno-reactivity towards isomerized and/or optically inverted antigens:

T-Cell Proliferation Assay

Antigen specific reactivity of T-cells isolated from either peripheral blood or from the affected target organ in the autoimmune diseases (i.e. synovial fluid/tissue of RA patients or the CNS of MS patients) or from animal models of the diseases can be measured by a lymphocyte proliferation assay. The lymphocytes are placed in culture in a suitable cell culture media, in the presence of either the specific antigen/antigen fragment in either βL, αD or βD form or with unrelated control antigen or no antigen at all. ³H-thymidine is added to the medium, and actively dividing lymphocytes stimulated by the presence of antigen will incorporate the labelled thymidine into the DNA. By quantifying the ³H-thymidine incorporated into the DNA, the proliferative response to the different forms of the auto-antigen or auto-antigen derived epitopes can be assessed (Weir 1996). Antigen-specific proliferation is a hallmark of specific CD4+ T-cell reactivity.

Limited Dilution Assays

In order to obtain information about the ‘titre’ of cellular immune-reactivity directed against a given auto-antigen or epitope thereof in βL, αD, or βD (or γL or γD) form, a limited dilution assay can be performed. This assay is performed by adding varying numbers of lymphoid cells (i.e. from peripheral blood) to individual culture wells and stimulating antigen and antigen presenting cells or specific growth factors. After several days the wells are tested for a specific response to antigen, such as cytotoxic killing of target cells or specific proliferation. Each well that contained a specific T-cell will make a response to its target and from the Poisson distribution one can determine that when 37% of the wells with a given dilution of T-cells are negative, each well contained, on average, one specific T-cell at the beginning of the culture. By comparing the response in individuals showing an autoimmune reactivity against an isomerised and or racemised antigen (i.e. RA or MS patients) and control individuals, the difference in T-cell titer between the two populations can be assessed and used as a measure of the antigen specific expansion of the auto-reactive cells which have occurred in the individuals suffering from the autoimmune disease.

ELISPOT Assay

The ELISPOT assay can be use as a sensitive method to quantify the single lymphocytes from i.e. a peripheral blood sample for production of specific antibodies (B-cells) or cytokines characteristic stimulated antigen specific T-cells. The ELISPOT assay is performed by culturing lymphocytes isolated from either peripheral blood or from the affected target organ in the autoimmune diseases (i.e. synovial fluid/tissue of RA patients or the CNS of MS patients) or from animal models of an autoimmune disease. The ELISPOT assay is performed by culturing the lymphocytes in a suitable culture-medium on a nitro-cellulose membrane or another solid surface capable of retaining proteins and peptides secreted by the lymphocytes (Ronnelid & Klareskog, 1997). When a given antigen is added to the culture medium, lymphocytes specific for this antigen or epitopes thereof will be stimulated and secrete characteristic lymphokines (i.e. interferon-γ, interleukin-2 or interleukin-4)(Weir 1996, Okamoto et al 1998). After a given period of culturing, cells are washed off the membrane and specific agents (i.e. antibodies) can be used to detect the lymphokines produced by the cells. By quantifying the number of cells producing a given lymphokine, as well as the pattern of lymphokine production, the response to the antigen used for stimulation can be assessed and characterised.

The invention will be further described and illustrated with reference to the following examples in which reference is made to the accompanying drawings, the content of which is as follows:

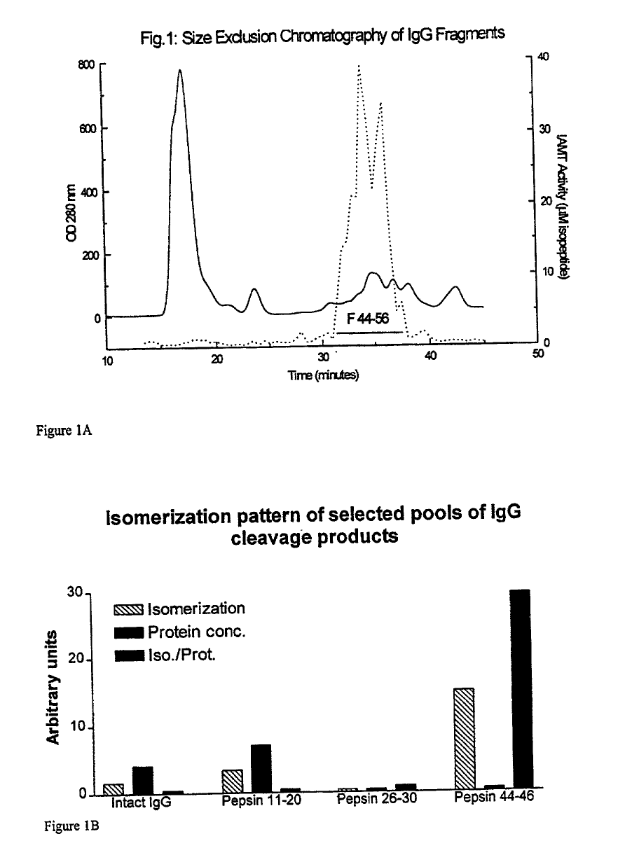

FIG. 1: (A) Shows the results obtained in example 1 from the first size exclusion chromatography of pepsin degraded human IgG in form of a graph showing OD280 nm of eluted material, as well as the IAMT reactivity measured in collected fractions. (B) shows the specific degree of isomerisation for the different fractions of pepsin degraded IgG isolated by size exclusion chromatography;

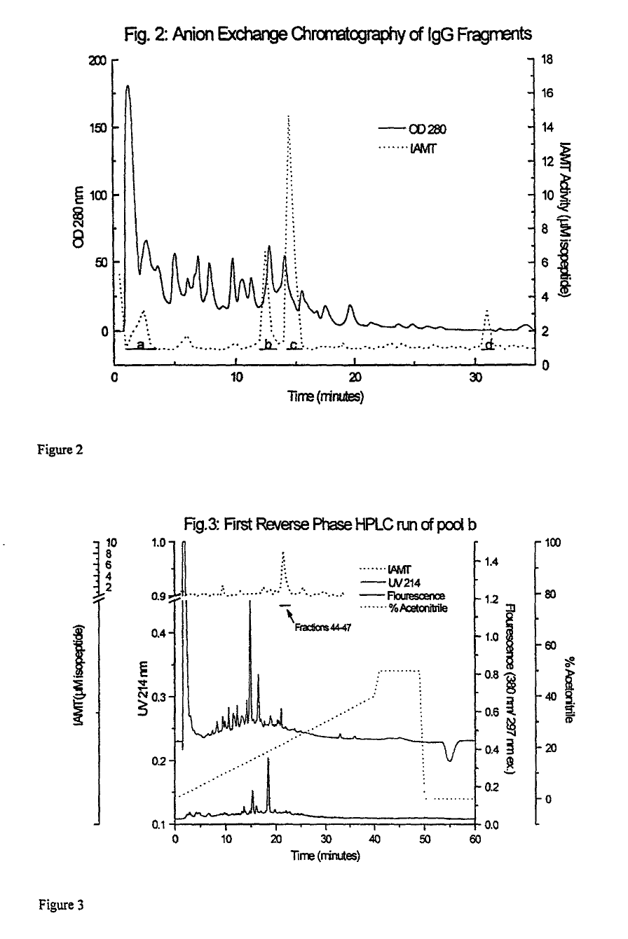

FIG. 2: Shows the results obtained in example 1 by subjecting the low molecular weight IgG fragments isolated by size exclusion chromatography to separation on an anion exchange column. Collected pools of fractions subjected to further purification are indicated as a, b, c and d;

FIG. 3: Shows the result from RP-HPLC separation of peptides from ‘pool b’ of the anion exchange purified IgG peptides depicted in FIG. 2. The following traces are shown: UV 214 nm, Fluorescence (380/297 nm), acetonitrile gradient, and IAMT reactivity;

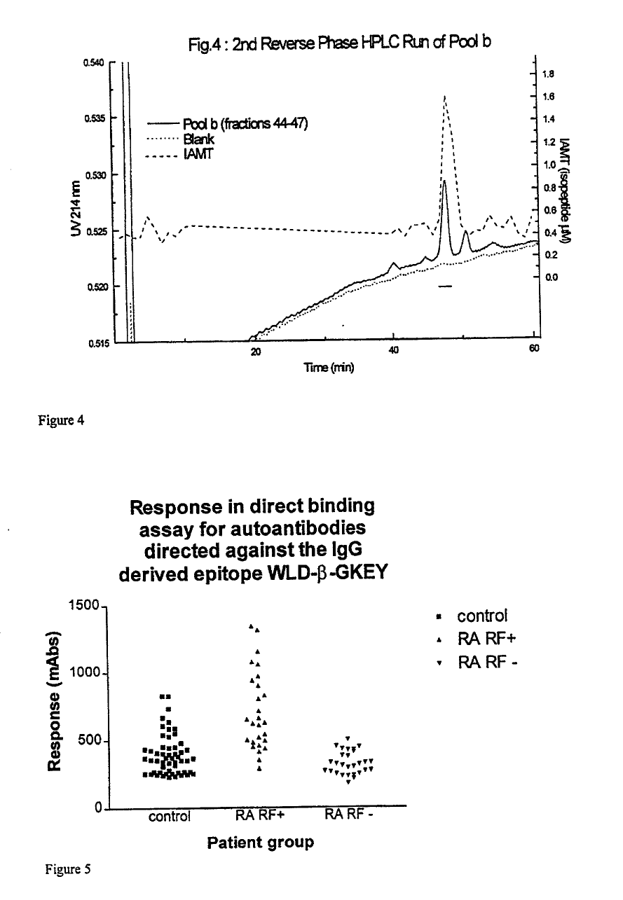

FIG. 4: Shows the result from a second round of RP-HPLC purification of the pool b purified as outlined in FIG. 3. The UV 214 nm detector signal as well as the IAMT reactivity of the eluted material is shown;

FIG. 5 shows results obtained in Example 2 in the form of a graph of signal obtained in an ELISA assay of serum samples from three patient groups;

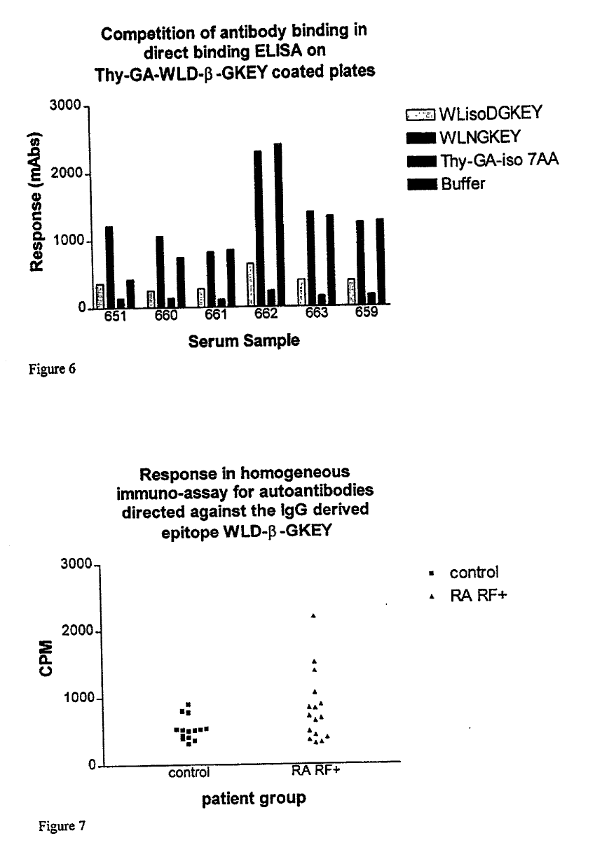

FIG. 6 shows results obtained in Example 3 in the form of a bar graph of ELISA signal for six serum samples tested in the presence of competing peptides;

FIG. 7: Show the results obtained in example 4 in the form of a graph of signal (in CPM) obtained in a homogeneous RIA assay with samples from RA patients and healthy controls;

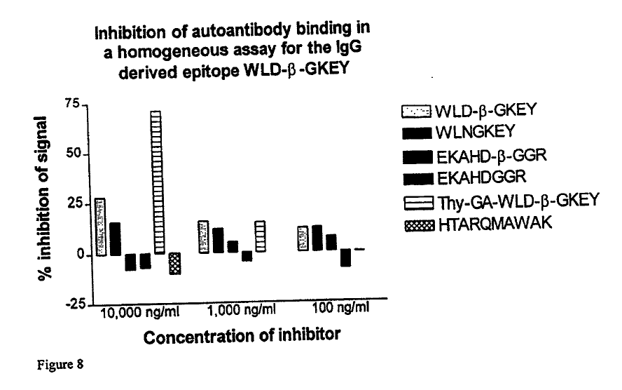

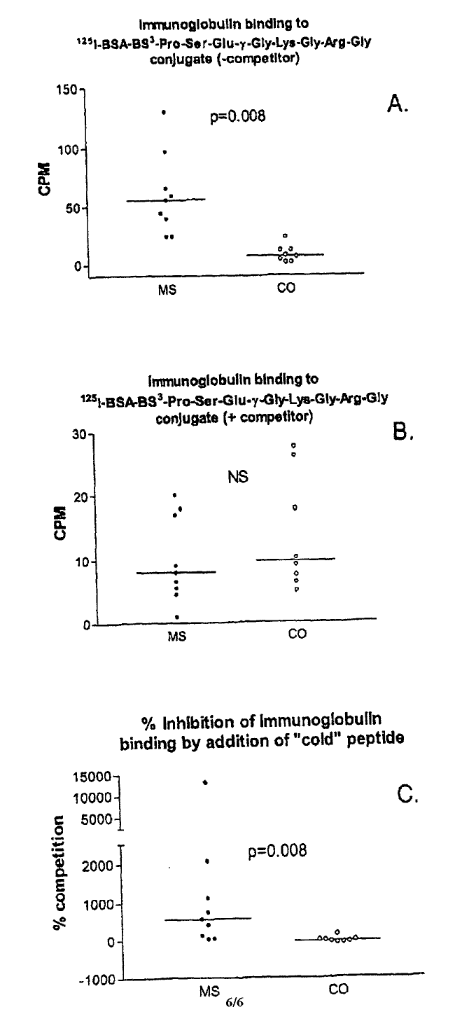

FIG. 8: Shows the results obtained in example 4 in the form of a bar graph of RIA signal expressed as percent inhibition obtain in the presence of competing peptide; and

FIG. 9: Shows results obtained in Example 6 in the form of scatter graphs A, B and C.

### EXAMPLE 1

Identification of Asn-315 of the Fc Region of IgG as Isomerisation Susceptible.

Human IgG (Sigma cat no. I-4506) was digested with pepsin according to the following protocol: The digestion is carried out with immobilised pepsin (Pierce Cat. No. 20343) essentially according to the procedures described by the manufacturer (Pierce). In short 0.125 ml of immobilised pepsin gel is added to a test tube and equilibrated in 0.5 ml of digestion buffer (20 mM sodium acetate buffer, pH 4.5). 10 mg of pure lyophilised IgG is added in 1.0 ml of digestion buffer, and the mixture is incubated in at 37° C. for four hours. Digestion is stopped by adding 1.5 ml of 10 mM Tris HCl, pH 7.5 to the incubation mixture. IgG fragments are subsequently separated from immobilised pepsin gel by centrifugation (1000 g for five minutes) and removing the supernatant containing the fragments.

The IgG fragments are separated from undigested IgG by gel-filtration on a Superdex 75 HR10/30 column (Pharmacia, Sweden). The column (2.6×72 cm (360 mL total volume)) is equilibrated in 0.2 M NH₄HCO₃ pH 8.0 at 28 ml/h. 2.75 ml sample is loaded and 0.25 mL fractions are collected. The column was calibrated with a mixture of the following Mw markers to allow size determination of eluted fragments: Albumin (67 kDa), Ovalbumin (43 kDa), Chymotrypsinogen A (25 kDa), Ribonuclease A (13.7 kDa) and Aprotinin (6.5 kDa). Low molecular weight (with a molecular weight below 10 kDa) fragments of IgG derived from the Fc portion of IgG appear in the elution volume 22-28 ml (fractions 44-56, FIG. 1).

Aliquots of these fractions are re-dissolved in phosphate buffered saline (PBS) and analysed for the presence of isomerised or optically inverted Asx residues by an enzyme assay with the enzyme L-iso-aspartyl(D-aspartyl) methyl-transferase (IAMT). Briefly described this assay is based on detection of isomerised residues by labelling with radioactive (tritiated) methionine by the IAMT enzyme. The assay is carried out as follows:

In a 600 μl Eppendorf tubes the following reagents are added: 15 μl bovine red blood cell lysate containing the IAMT activity (prepared according to Murray and Clarke 1984), 10 μL assay buffer (0.25 M NaH₂PO₄/NaOH pH 7.02), 15 μl sample (or calibrators made up of synthetic isomerised peptide solutions of known concentration) and 10 μl SAM tracer (prepared as follows: 3 ml cold SAM is added to 26.1 ml freshly prepared 10 mM HCl. To 20 ml of this solution 100 μl “Hot SAM-” (Amersham TRA236, 1000 μmol/L) is added, and the solution is stored in 1 ml aliquots at −18° C.). After whirly mixing the vials are incubated for 60±1 min. at 37° C. (on a water-bath). The reaction is stopped by addition of 50 μl Quenching solution (0,2 M NaOH, 1% Sodium dodecyl sulphate), followed by mixing. 75 μl of this solution is spotted onto a filter-paper (0.75×5.5 cm pre-folded in “accordion-pleats”). The filter paper is placed in 6 ml scintillation tubes containing 2.5 ml Ecoscint H scintillation fluid (submersed approximately 1.5 cm in the tube. The tubes are left at room temperature for approximately 18 hours (over-night) in order to allow radioactive methanol to diffuse into the scintillation fluid. The filter strips are removed and the vials are counted in a β-counter with the following stop conditions: 900 sec., or a maximum of 6400 CPM. The concentration in unknown samples are calculated from the standard curve prepared from the measurements of the calibrators made up of synthetic iso-peptides of known concentration.

These measurements demonstrated that the low molecular weight fractions from the pepsin degradation of IgG have a high degree of IAMT activity, apparently containing the majority of the isomerised sites of the intact IgG molecule (fractions 44-56, FIG. 1). These fractions are further purified by reverse phase high pressure liquid chromatography (RP-HPLC).

The fractions from the size exclusion chromatography column containing the IAMT reactive peptides (Fractions 44 to 56) were pooled, the volume was adjusted to 9.5 ml and 20 μl TFA was added. A Sep-Pak C18 cartridge (3 cc, 500 mg, Waters) was conditioned with 10 ml 80% Methanol and equilibrated with 10 ml 0.2% TFA. The sample was applied to the column, and the column was washed with 20 ml 1% TFA. Finally the bound peptides were eluted with 10 ml 40% acetonitrile, 0.1% TFA. Eluents were collected, frozen and lyophilised. Eluents were re-dissolved in 2 ml 20 mM Tris-buffer pH 7.88. 100 μl of this solution was stored for measurement of IAMT activity, the rest (1900 μl) was used for ion exchange chromatography.

The anion exchange chromatography was performed using a 1 ml mono-Q HR 5/5 column (Pharmacia 52-1622-00). The column was equilibrated with 20 mM Tris, pH 7.88 at a flow-rate of 1 ml/min. The sample was loaded via a manual injector loop and the column was eluted using a linear NaCl gradient (0.0-0.3M NaCl, applied over 30 minutes). Then a linear gradient from 0.3 to 1 M NaCl was applied over 1 minute. Elution was continued for 1 minute with 1 M NaCl, and finally a linear gradient from 1 M to 0 M NaCl was applied over 1 minute. Elution was continued with this buffer for 2 minutes. Eluted peptides were detected by TV-absorption at 280 nm and 0.5 ml fractions (30 sec) were collected. Aliquots of the fractions were analysed in the IAMT assay and the results are plotted in FIG. 2. Fractions 4-6 were pooled (a), fractions 25-27 were pooled (b), fractions 29-31 were pooled (c) and stored for further fractionation.

The pooled fractions were buffer changed using a Sep-Pak C18 cartridge as described above, re-dissolved in 200 μl 0.1 % w/w trifluoroacetic acid (TFA) and purified further by RP-HPLC. The first round of RP-HPLC is performed on a C-18 column (Nova-Pak C-18 4 μm 3.9×150 mm HPLC column, Waters) with a linear gradient from 0 to 40% acetonitrile over 40 min in 0.1% (w/w) trifluoroacetic acid (TFA) with a flow of 1 ml/min. Eluted peptides were detected by UV-absorption at 214 nm and by fluorescence (at 380 nm (emission) using 297 nm light for excitation) and 0.5 ml fractions (30 sec.) were collected and lyophilised for analysis in the IAMT assay.

For pool ‘a’ from the anion exchange column, the majority of the IAMT reactive material is eluted between 22 and 23.5 min (FIG. 3). These fractions are pooled, the peptides in the fractions were concentrated by use of a Sep-Pak C-18 column as described above. The sample was re-dissolved in 200 μl 0.05% heptafluorobutyric acid (HFBA). This sample is further purified by a second round of RP-HPLC on the same column, but this time performing a linear gradient from 5-30% acetonitrile in 0.05% heptafluoro-butyric acid (HFBA) over 80 min with collection of 0.5 ml fractions. The fractions were monitored by UV-absorption at 214 nm and by fluorescence (at 380 nm (emission) using 297 nm light for excitation) and the lyophilised fractions were re-dissolved for analysis in the IAMT assay.

As is apparent from FIG. 4, one major peak from the second round of HPLC purification of pool ‘b’ from the anion exchange column (FIG. 2), contains the majority of the isomerised Asx residues (FIG. 4). The fraction containing this peak was subjected to amino-acid sequencing, using an Applied Biosystems model 477A sequencer according to the manufacturers instructions. The following sequences are deduced:

‘pool b’: His-Gln-Asp-Trp-Leu
‘pool c’: Thr-Val (Leu/Val)His-Gln-Asp-Trp-Leu-Asp-Gly-Lys-
Glu-Tyr-Lys-Cys-Lys-Val-Ser-Asn-Lys (Ala/Gly) Leu-Pro
C_{H}2: Thr-Val (Leu/Val)His-Gln-Asp-Trp-Leu-Asn-Gly-Lys-
Glu-Tyr-Lys-Cys-Lys-Val-Ser-Asn-Lys (Ala/Gly) Leu-
Pro (Ala/Ser) (Pro/Ser) Ile-Glu-Lys-Thr-Ile-Ser-Lys (Ala/Thr) Lys

Thus, the isolated isomerised peptides are derived from the C_{H}2 region but processed to different length by the pepsin degradation. The published sequence for the C_{H}2 region is given above, and it is apparent that all three peptides match this sequence. The asparagine residue given in bold is Asn-315. Residue 308 is a valine in IgG₂ and a leucine in the other IgG subclasses. The sequence of the ‘pool c’ peptide given in italics is inferred from amino acid analysis (see below). Residue 315 is there given as an aspartic acid instead of an asparagine in accordance with the peptide bond between this residue and the succeeding glycine being rearranged from the α- to the β-carboxyl group.

All isolated peptides are recognised by the iso-aspartate specific IAMT enzyme and they can not be sequenced beyond the leucine residues N-terminal to the Asn-315 residue. This strongly suggests that the Asn-315 residue has undergone an isomerisation reaction whereby the peptide bond has been re-arranged from the normal α-carboxyl group the β-carboxyl group of the side chain, concomitant with the hydrolytic removal of the amino group. The inability to sequence the peptides beyond residue 315 is in accordance with previous results demonstrating that such an isomerisation reaction results in inability to sequence the peptide beyond the susceptible site by normal Edman degradation (Fledelius et al 1997).

### EXAMPLE 2

Detection of Auto-Antibodies Reactive with Asn-315.

It was investigated whether auto-antibodies would recognise an Asn-315 derived septa-peptide and whether such auto-antibodies are a characteristic feature of rheumatoid arthritis. In order to carry out such investigations the IgG Fc Asn-315 derived (βL) septa-peptide Trp-Leu-Asp-β-Gly-Lys-Glu-Tyr, was manufactured synthetically. The peptide used was a preparation of linear (αL form) Trp-Leu-Asn-Gly-Lys-Glu-Tyr peptide heated to promote isomerisation by the following procedure. The peptide was dissolved in the buffer and heated for 4 hours at 90° C. to promote isomerisation and/or optical inversion. The resulting mix of isomerised (βL) optically inverted (αD and βD) and linear (αL) peptides were analysed by RP-HPLC using a 15-35% acetonitrile gradient in 0.1% TFA over 10 minutes with a flow rate of 1 ml/min, and the isomerisation status of the resulting peaks were studied by amino-acid analysis as described above. This peptide was dissolved at 5 mg/ml in 0.2 M Na-Phosphate pH 9.2. The peptide was coupled to thyroglobulin (Sigma lot 66H7085) with glutaraldehyde (GA) (Fluka 49626 lot 43381/1) using the following protocol.

0.5 ml thyroglobulin (30 mg/ml) in 0.1 M Na-phosphate buffer pH 8.0, was added drop-wise (over 2 minutes) and under constant mixing to 0.5 mL of the following solution: 10% GA, 40% H₂O, 50% 0.2 M Na-Phosphate pH 8.0. The vial was incubated overnight with mixing at room temperature. Excess GA was removed by gel filtration (NAP-lo column, Pharmacia), and the buffer changed to PBS. The final volume was adjusted to 1.5 mL (10 mg/mL carrier-protein of each preparation). 500 μl of carrier protein was incubated with 500 μL 5 mg/ml peptide solution. The vials were incubated for 24 hours at room temperature and under constant mixing. Excess peptide was removed by gel filtration (NAP-10 column, Pharmacia) into PBS buffer. The final volume was adjusted to 1500 μL and the protein concentration was determined by a BioRad protein assay performed according to the manufacturers instructions.

The Thy-GA-Trp-Leu-*Asx-Gly-Lys-Glu-Tyr conjugate was dissolved in PBS to a final concentration of 10 μg/ml, and 100 μL of this solution is pipetted into the wells of a micro-titre-plate (MTP, flat-well polysorb, Nunc). The plate was blocked as described (Bonde et al 1994), and serum samples diluted one-hundred fold in 10 mM Na-phosphate, 140 mM NaCl, 0.1% tween-20, 1% BSA pH 7.4 (assay buffer) were added. The MTP was left for one hour ±5 minutes on a rotary shaker at 20° C. The plates were washed five times in washing buffer (25 mM tris, 140 mM NaCl, 0.1% tween-20 pH 7.4), by a manual plate washer. One-hundred μL assay buffer containing a one-thousand fold dilution of Peroxidase conjugated rabbit anti human λ-chains (Dako 063) and κ-chains (Dako 013) was added into each well. The MTP was again incubated for one hour ±5 minutes on a rotary mixer at 20° C. After 5 times washing, 100 μl of peroxidase substrate was added (3,3′,5,5′ tetra-methylbenzidine dihydrochloride (TMB), Kirkegaard & Perry Laboratories, USA) and incubated for 15±2 minutes at room temperature (18-22° C.) in the dark. After addition of 0.18 M H₂SO₄, the absorbency was measured at 450 nm.

When RA sera positive for rheumatoid factor, RA sera negative for rheumatoid factor and control sera are compared, a significantly elevated reactivity is detected in the RA RF+ population compared to the other two populations (FIG. 5). Experiments performed in parallel, following the same assay procedure but using MTP coated with another irrelevant Thy-GA conjugate (Thy-GA-Glu-Lys-Ala-His-*Asx-Gly-Gly-Arg) showed no differentiation between the groups. This demonstrates that in RA patients positive for RF, an elevated auto-antibody reactivity towards the IgG Fc Asn-315 derived sequence Trp-Leu-*Asx-Gly-Lys-Glu-Tyr exists.

### EXAMPLE 3

Competition of the Binding of Specific Auto-Antibodies Recognising Isomerised Asn-315 with Synthetic Septides.

The binding of RA autoantibodies to the Thy-GA-Trp-Leu-*Asx-Gly-Lys-Glu-Tyr coated plates as described above could be competed out by pre-incubating the sera with Trp-Leu-*Asx-Gly-Lys-Glu-Tyr peptide in solution. The experiment was carried out as follows. Serum samples were diluted one-hundred fold in 10 mM Na-phosphate, 140 mM NaCl, 0.1% tween-20, 1% BSA pH 7.4 (assay buffer). Two-hundred μl was added to 1.5 ml polypropylene tubes followed by addition of 50 μl of the following reagents dissolved in assay buffer at a concentration of 50 μg/ml:

1. Trp-Leu-Asp-β-Gly-Lys-Glu-Tyr. (βL form of the epitope)

2. Trp-Leu-Asn-Gly-Lys-Glu-Tyr (αL form of the epitope)

3. Thy-GA-Trp-Leu-*Asx-Gly-Lys-Glu-Tyr conjugate (prepared as described in example 2).

4. Assay buffer alone.

The tubes were placed at 4° C. for 17 hours, and one-hundred μl of the mixture was added into MTP coated with Thy-GA-Trp-Leu-*Asx-Gly-Lys-Glu-Tyr conjugate. The assay was performed as described above under example 2.

The results are shown in FIG. 6. It is apparent that the isomerised peptide (1) as well as the Thy-GA-Trp-Leu-*Asx-Gly-Lys-Glu-Tyr conjugate (3) are able to compete out the binding of 6 analysed RA sera. This strongly suggests, that auto-antibodies among the RA patients recognising the seven amino acid epitope surrounding residue 315, predominantly are specific for the isomerised form (βL) of the epitope.

### EXAMPLE 4

A Homogeneous Radio-Immuno Assay for Assessment for Detection of Auto-Antibodies Reactive with Isomerised IgG Fc Asn-315 Derived Peptides.

A homogeneous RIA assay was developed for measurement of auto-antibodies with reactivity towards the isomerised form of the epitope Trp-Leu-Asn-Gly-Lys-Glu-Tyr derived from the IgG C_{H}² region. The assay was performed by incubating serum samples with ¹²⁵I Thy-GA-Trp-Leu-*Asx-Gly-Lys-Glu-Tyr over-night followed by precipitation of immune-complexes with protein A Sepharose.

Serum samples are diluted 1:200 in IMP buffer (IMP-buffer: 10 mM Na-Phosphate, pH 7.4, 140 mM NaCl, 5 mM EDTA, 0.5% Triton X-100, 0.1% BSA, 10 μg/ml soy-bean trypsin inhibitor). A Thy-GA-Trp-Leu-*Asx-Gly-Lys-Glu-Tyr conjugate is prepared as described in example 2 and iodinated with ¹²⁵I using the chloramine T protocol: 100 μg of the conjugate is diluted in 0.25 M Na₂HPO₄ to a total volume of 140 μl. 1.5 mCi Na¹²⁵I is added followed by 10 μL Chloramin-T (1 mg/ml, prepared freshly). The solution is vortexed for 30 seconds and 150 μL methionine (1 mg/ml) is added and immediately vortexed for 120 sec. The tracer is purified on size exclusion column (type: BIOSEP SEC S-2000, size: 300×7.80 mm) equilibrated with PBS containing 1% BSA Flow rate 1.0 ml/min. 500 μl fractions are collected and analysed in a scintillation counter (γ-counter). The fractions containing the tracer are pooled and used in the homogeneous RIA assay format.

The ¹²⁵I Thy-GA-Trp-Leu-*Asx-Gly-Lys-Glu-Tyr tracer is diluted in IMP-buffer and seventy-five μl of 200× diluted serum sample is mixed with 25 μl peptide/streptavidin solution in a sealed polypropylene vial. The vial is incubated over night (16-18 hours) at 4° C. Protein A Sepharose (PAS) (20 μl/sample vial) is weighed out and washed 3 times with 10 ml IMP washing buffer, and transferred to Eppendorf 1.5 ml tubes using a repeater pipette. The PAS is sedimented by centrifugation at 1000 RPM 2 min., and the supernatant is aspirated using a suction flask or a pipette. After the three hour incubation the antibody/antigen solution is transferred to the PAS pellets, and incubated for an additional 30 min at room temperature on a shaking table. The PAS is sedimented by centrifugation at 1000 RPM for 2 min. The PAS pellets are washed 5 times with 750 μl IMP washing buffer. After each washing step the PAS is sedimented by centrifugation at 1000 RPM 2 min, and the supernatant is aspirated using a suction flask or a pipette. Finally the PAS pellets are re-suspended to a 100 μL slurry in milli-Q water and transferred to 4 ml Polypropylene tubes for counting in the γ-counter. As described for the heterogeneous ELISA assay format above, control experiments will be carried out with non-sense peptides.

Practising according to this protocol, a high specific signal was obtained with sera. On a group basis, RF+RA patients showed a higher response than serum samples from control subjects (FIG. 7).

Competition experiment was performed by adding 25 μl of solutions containing 100, 1000 or 10,000 ng/ml of the following peptides: Trp-Leu-Asp-β-Gly-Lys-Glu-Tyr, Trp-Leu-Asn-Gly-Lys-Glu-Tyr, Glu-Lys-Ala-His-Asp-β-Gly-Gly-Arg and Glu-Lys-Ala-His-Asp-Gly-Gly-Arg peptides as well as a ‘non-sense’ control peptide His-Thr-Ala-Arg-Gln-Met-Ala-Trp-Ala-Lys and the Thy-GA-Trp-Leu-*Asx-Gly-Lys-Glu-Tyr and Thy-GA-Glu-Lys-Ala-His-*Asx-Gly-Gly-Arg conjugates. When the reactivity observed in all experiments is compiled, and the reactivity is calculated on a group basis, a significant reactivity (P=0.01 by Student's T-Test) is seen towards the Trp-Leu-Asp-β-Gly-Lys-Glu-Tyr peptide in the highest concentration (and with the Thy-GA-Trp-Leu-*Asx-Gly-Lys-Glu-Tyr conjugate), but not with any of the other peptides (FIG. 8). This is similar to observations made in the ELISA formats described in the previous examples.

### EXAMPLE 5

Mapping of an Immuno-Reactive Epitope Containing the Asn-315 of Human IgG, Reactive with an Immuno-Affinity Purified Autoantibody from an RA Patient.

Analysis of IgM auto-antibodies opens up the possibility of removing endogenous IgG from the serum samples prior to analysis for auto-antibodies. In fact, it has been conclusively established that significant amounts of circulating isomerised/optically inverted IgG is present in both normal and RA individuals, and it is entirely conceivable that any putative anti iso-IgG auto-antibody may be almost entirely blocked by the presence of these molecules, thus hampering the binding to the solid phase in the assays. The following example describes a method for purification of human auto-antibodies reactive with isomerised target epitopes derived from human IgG, and detection of reactivity towards such epitopes demonstrated by incubation with synthetic peptides synthesised on an inactive cellulose support.

Purification of RF's from Human Serum Samples by Immuno-Affinity Chromatography on an IgG Column.

The experimental protocol that was applied is as follows:—

1. IgG is coupled to CNBr activated Sepharose using the manufacturers instructions (Pharmacia, Upsala, Sweden).

2. The IgG Sepharose is packed in a suitable column (i.e. a disposable DG10 column, BioRad laboratories, Richmond, Calif.). The column is washed with at least 10 column volumes PBS and with 10 column volumes 0.1 M sodium acetate, 0.15 M sodium chloride pH 3.5 and finally equilibrated in PBS.

3. Serum from RA patients is diluted 10 times in PBS and loaded on the column. The column is washed with PBS until the absorbency (OD 280 nm) reaches base-line.

4. Bound RF's are eluted with 0.1 M sodium acetate, 0.15 M sodium chloride pH 3.5.

5. IgM is separated from IgG and IgA by gel-filtration on a Sephadex G-200 column in 0.1 M sodium acetate, 0.15 M sodium chloride pH 3.5.

6. Eluted IgM is diluted in PBS-BT to 1 μg/mL and assayed as described below.

Synthesis of Cellulose-Bound Peptides

Seven amino-acid peptides extending over the Asn-315 residue were synthesised by spot synthesis using Whatman 540 paper (Maidstone, U.K.), using the previously described procedures (Frank, 1992, Kramer et al 1994). The peptides were automatically prepared using a spot synthesis method (Abimed, Langenfeld, F R G). The following peptides were synthesised, covalently attached via the carboxy-terminus:

I. Thr-Val-Leu-His-Gln-Asp-Trp . . .
II. Val-Leu-His-Gln-Asp-Trp-Leu . . .
III. Leu-His-Gln-Asp-Trp-Leu-Asp-B . . .
IV. His-Gln-Asp-Trp-Leu-Asp-β-Gly . . .
V. Gln-Asp-Trp-Leu-Asp-β-Gly-Lys . . .
VI. Asp-Trp-Leu-Asp-β-Gly-Lys-Glu . . .
VII. Trp-Leu-Asp-β-Gly-lys-Glu-Tyr . . .
VIII. Leu-Asp-β-Gly-Lys-Glu-Tyr-Lys . . .
IX. Asp-β-Gly-Lys-Glu-Tyr-Lys-Cys . . .
X. Gly-Lys-Glu-Tyr-Lys-Cys-Lys . . .

Asp-β:

denominates an isomerised Asp residue, where the peptide linkage goes through the β-carboxyl instead of the normal α-carboxyl group (βL form).

. . . :

denominates attachment to the cellulose support.

The cellulose membrane containing these peptides was incubated with the immunoaffinity purified RA auto-antibodies purified as described above. The incubation and visualisation of the bound antibodies was carried out using conventional immuno-blotting techniques as described (Kramer et al 1994).

The experiment demonstrated strong reactivity towards peptide IV and VII. This strongly suggests, that auto-antibodies purified from the RA patient serum are recognising a seven amino acid epitope surrounding residue Asn-315 in the isomerised (βL) form of the epitope.

### EXAMPLE 6

Detection of Auto-Antibodies Reactive with Pro-Ser-Glu-γ-Gly-Lys-Gly-Arg-Gly

It was investigated whether auto-antibodies in patients affected with multiple sclerosis would recognise the octapeptide Pro-Ser-Glu-γ-Gly-Lys-Gly-Arg-Gly derived from myelin base protein (MAP).

For this purpose the MBP derived octapeptide Pro-Ser-Glu-γ-Gly-Lys-Gly-Arg-Gly (γL) was manufactured synthetically using standard FMOC chemistry. The peptide was conjugated to bovine serum albumin (ISA) by Bis-[sulfosuccinimidyl]suberate(BS³) according to the manufacturers instructions (Pierce) and iodinated with ¹²⁵I using the chloramine T protocol as described in Example 4.

Serum from 9 MS-patients and 8 healthy persons are reacted overnight with ¹²⁵I—BSA-S³-Pro-Ser-Glu-y-Gly-Lys-Gly-Arg-Gly followed by precipitation of immune complexes with protein A Sepharose. Serum samples are diluted 1:200 in IMP buffer. The ¹²⁵I BSA-BS³-Pro-Ser-Glu-y-Gly-Lys-Gly-Arg-Gly tracer is diluted in IMP buffer (to an activity of 100000 CPM/25 μl). Seventy-five Al of diluted serum sample is mixed with 25 μl tracer and 25 μl of either IMP buffer or free (unconjugated/unlabelled) Pro-Ser-Glu-y-Gly-Lys-Gly-Arg-Gly peptide (in a concentration of 10 μg/ml in IMP buffer) is added to this mixture. This mixture incubated overnight at 4° C. Protein A Sepharose (PAS) 20 μl/sample vial is weighed out and washed 3 times with 10 ml IMP washing buffer, and transferred to eppendorf 1.5 ml tubes using a repeater pipette. The PAS is sedimented by centrifugation at 2000 RPM 2 min., and the supernatant is aspirated using a suction device (or a pipette). After the overnight incubation the antibody/antigen solution is transferred to the PAS pellets, and incubated for 3 hours at room temperature on a shaking table. The PAS is sedimented by centrifugation at 2000 RPM for 2 min. The PAS pellets are washed 5 times with 750 μl IMP washing buffer. After each washing step the supernatant is aspirated. Finally the PAS-pellets are re-suspended to a 100 μl slurry in milli-Q water and transferred to 4 ml Polypropylene tubes for counting in a γ-counter. Practising according to this protocol, a specific signal was obtained with sera. On a group basis MS sera showed a significantly higher response than serum samples from control subjects (P=0.0078, two-tailed non-parametric t-test), FIG. 9A. Moreover the binding of ¹²⁵I—BSA-BS³-Pro-Ser-Glu-γ-Gly-Lys-Arg-Gly conjugate to human immunoglobulin could be specifically inhibited by addition of “free, Pro-Ser-Glu-γ-Gly-Lys-Gly-Arg-Gly peptide. Thus the % inhibition of immunoglobulin binding was significantly higher (p=0.008, two-tailed non-parametric t-test) in MS sera as compared to control sera FIG. 9C. Hence MS patients have antibodies directed against a MBP derived epitope containing isomerised (γL) glutamic acid.

The results are shown in FIG. 9 in which the three panels are as follows:—

Panel A, binding of human immunoglobulin to ¹²⁵I—BSA-BS³-Pro-Ser-Glu-γ-Gly-Lys-Gly-Art-Gly (no free peptide added as competitor) in sera from patients with multiple sclerosis (MS) and healthy controls (CO).

Panel B, binding of human immunoglobulin to ¹²⁵I—BSA-BS³-Pro-Ser-Glu-γ-Gly-Lys-Gly-Art-Gly (free Pro-Ser-Glu-γ-Gly-Lys-Gly-Art-Gly peptide added as competitor) in sera from patients with multiple sclerosis (MS) and healthy controls (CO).

Panel C, percent inhibition of binding of human immunoglobulin to ¹²⁵I—BSA-BS³-Pro-Ser-Glu-γ-Gly-Lys-Gly-Art-Gly in sera from patients with multiple sclerosis (MS) and healthy controls (CO).

### REFERENCES

1. Aanstoot H J, Kang S M, Kim J, Lindsay L A, Roll U, Knip M, Atkinson M, Mose-Larsen P, Fey S, Ludvigsson J, Madsen O, & Baekkeskov S. J.Clin. Invest. (1996) June 15; 97(12);2772-83 Identification and characterisation of glima 38, a glycosylated islet cell membrane antigen, which together with GAD65 and IA2 marks the early phases of autoimmune response in Type I Diabetes

2. Abel L C, Kalil J, Cunha Neto E (1997) Braz. J. Med. Biol. Res. (1997) Molecular mimicry between cardiac myosin and Trypanosoma cruzi antigen B13: identification of a B13-driven human T cell clone that recognizes cardiac myosin. 30(11):1305-1308

3. Adelmann M, Wood J, Benzel I, Fiori P, Lassman H, Matthieu J -M, Gardinier M V, Dornmair K, Linnigton C (1995) The N-terminal domain of the myelin oligodendrocyte glycoprotein (MOG) induces acute demyelinating experimental autoimmune encephalomyelitis in the Lewis rat. J Neuroimmunol. 63: 17-27

4. Atassi M Z, Oshima M (1997) Autoimmune responses against acetylcholine receptor: T and B cell collaboration and manipulation by synthetic peptides. Crit. Rev. Immunol. 17(5-6):481-495

5. Bach-Elias M, Bahia D, Teixeira D C, Cicarelli R M (1998) Presence of autoantibodies against small nuclear ribonucleoprotein epitopes in Chagas' patients' sera. Parasitol Res. 84(10) :796-799

6. Beniac D R, Luckevich M D, Czarnota G J, Tompkins T A, Ridsdale R A, Ottensmeyer F P, Moscarello M A, Harauz G (1997) Three-dimensional structure of myelin basic protein. I. Reconstruction via angular reconstitution of randomly oriented single particles. J. Biol. Chem. 272(7): 4261-4268.

7. Benkirane N, Friede M, Guichard G, Briand J -P, Van Regenmorte M H V, Muller S. (1993) Antigenicity and immunogenicity of modified synthetic peptides containing D-amino acid residues. Antibodies to a D-enantiomer do recognize the parent L-hexapeptide and reciprocally J.Biol Chem 268: 26279-26285

8. Bernstein R M (1990) Humoral autoimmunity in systemic rheumatic disease, A review. J Royal Col. Phys. London. 24: 18-25

9. Bettadapura J, Menon K K, Moritz S, Liu J, Bernard C C A (1998) Expression, purification and encephalitogenicity of recombinant human myelin oligodendrocyte glycoprotein J Neurochem 70: 1593-1599

10. Baekkeskov S, Nielson J H, Marner B, Bilde T, Ludvigsson J, Lernmark A (1982) Autoantibodies in newly diagnosed diabetic children immunoprecipitate human pancreatic islet cell proteins Nature 298:167-169

11. Baekkeskov S, Aanstoot H -J, Christgau S, Reetz A, Solimena M, Cascalho M, Folli, F, Richter-Olesen H, and De-Camilli P (1990) Identification of the 64K autoantigen in insulin-dependent diabetes as the GABA-synthesizing enzyme glutamic acid decarboxylase Nature 347(6289):151-156

12. Berrih-Aknin S, (1995) Myasthenia gravis, a model of organ-specific autoimmune disease. J. Autoimmun 8(2):139-143

13. Bonagura V R, Artandi S E, Davidson A, Randen I, Agostino N, Thompson K, Natvig J T, Morrison S L (1993) Mapping studies reveal unique epitopes on IgG recognized by rheumatoid arthritis-derived monoclonal rheumatoid factors. J Immunol. 151: 3840-3852

14. Bonde M, Qvist P, Fledelius C, Riis B J, C. Christiansen C. 1994 Immunoassay for quantifying type I collagen degradation products in urine evaluated. Clin. Chem. 40, 2022-2025

15. Bonifacio E, Lampasona V, Genovese S, Ferrari M, Bosi E, (1995) Identification of protein tyrosine phosphatase-like IA2 (islet cell antigen 512) as the insulin-dependent diabetes-related 37/40K autoantigen and a target of islet-cell antibodies. J. Immunol. 155(11):5419-5426

16. Carson D A (1993). In: Kelley W N, Harris E D, Ruddy S, Sledge C B (Eds.). Textbook of Rheumatology; 1: 155-163, W B Saunders, London.

17. Chen K R, Shimizu S, Miyakawa S, Ishiko A, Shimizu H, Hashimoto T (1996) Coexistence of psoriasis and an unusual IgG-mediated subepidermal bullous dermatosis: identification of a novel 200-kDa lower lamina lucida target antigen. Br J Dermatol 134(2): 340-346

18. Chen P, Fong S, Carson D A. (1987) Rheumatoid factor Rheum. Dis. Clin. North Am. 13: 575.

19. Clarke S (1987). Propensity for Spontaneous Succinimide Formation from Aspartyl and Asparaginyl Residues in Cellular Proteins. Int. J. Peptide Protein Res. 30: 808-821.

20. Cooke A (1988) Mechanisms of autoimmunity. Diabetic Med. 5: 782-788

21. Dean B M, Becker F, McNally J M, Tarn A C, Schwartz G, Gale E A, Bottazzo G F(1986) Insulin autoantibodies in the pre-diabetic period: correlation with islet cell antibodies and development of diabetes. Diabetologica 29:339-342

22. De Vries H E, Kuiper J, De Boer A G, Van Berkel T J C and Breimer D D, Pharmacological Reviews (1997) Vol. 49, No.2, 143

23. Dieterich W. Ehnis T, Bauer M, Donner P, Volta U, Riecken E O, Schuppan D (1997) Identification of tissue transglutaminase as the autoantigen of celiac disease. Nature Med. 3(7):797-801

24. Dieterich W, Laag E, Schopper H, Volta U, Ferguson A, Gillet H, Riecken E O, Schuppan D (1998) Autoantibodies to tissue transglutaminase as predictors of celiac disease Gastroenterology 115(6):1317-1321

25. Fledelius C, Johnsen A, Cloos P, Bonde M, Qvist P. 1997 Characterisation of urinary C-terminal telopeptide degradation products. Identification of a β-isomerised Aspartyl residue in the C-terminal α₁ chain of type I collagen. J. Biol. Chem. 272: 9755-9763.

26. Frank, R (1992) Spot synthesis: An easy technique for the positionally addressable, parallel chemical synthesis on a membrane support. Tetrahedron 48: 9217-9232

27. Galletti P, Ingrosso D, Manna C, Clemente G, Zappia V. (1995) Protein Damage and Methylation—mediated repair in the erythrocyte Biochem J. 305: 313-325

28. Geiger T, Clarke S, (1987) Deamidation, Isomerization, and Racemization at Asparaginyl and Aspartyl Residues in Peptides. J. Biol. Chem. 262: 785-794

29. Glant T T, et al Mapping of Arthritogenic/Autoimmune Epitopes of Cartilage Aggrecans in Proteoglycan-Induced Arthritis. Scand J Rheumetol 1995: 24 (Suppl.101) 43-49.

30. Goin J C, Leiros C P, Borda E, Serin-Borda L (1997) Interaction of human chagasic IgG with the second extracellular loop of the human heart muscarinic acetylcholine receptor: functional and pathological implications FASEB J. 11(l) :77-83

31. Gorsuch A N, Spencer K M, Lister J, McNally J M, Dean B M, Bottazo G F, Cudworth A H. (1981) Evidence for a long prediabetic period in type I (insulin-dependent) diabetes mellitus. Lancet 2(8260-61): 1363-1365

32. Gottlieb S L, Gilleaudeau P, Johnson R, Estes L, Woodworth T G, Gottlieb A B, Krueger J G (1995) Response of psoriasis to a lymphocyte-selective toxin (DAB389IL-2) suggests a primary immune, but not keratinocyte, pathogenic basis. Nat.Med.1(5) :442-447

33. Harris E D (1993) Aetiology and pathogenesis of Rheumatoid Arthritis In: Kelley W N, Harris E D, Ruddy S, Sledge C B (Eds.). Textbook of Rheumatology; 1: 833-873, W B Saunders, London.

34. Ichikawa M, Johns T G, Jiu J, Bernard C C (1996) Analysis of the fine B cell specificity during the chronic/relapsing course of a multiple sclerosis-like disease in Lewis rats injected with the encephalitogenic myelin oligodendrocyte glycoprotein peptide 35-55. J. Immunol. 157:919-926

35. Inman R D, Day N K (1981). Immunologic and Clinical Aspects of Immune Complex Disease. Am. J. Med. 70: 1097.

36. Janeway C A Jr. Travers P, Hunt S, “Immuno Biology” (1997) 3^{rd} ed. Current Biology Ltd. (London) pp 226-235.

37. Johnson P M, Page Faulk W (1976). Rheumatoid Factor: Its Nature, Specificity, and Production in Rheumatoid Arthritis. Clin. Immun. Immunopath. 6: 414-430.

38. Johnson P M, Watkins J, Scopes P M, Tracey B M (1974). Differences in Serum IgG Structures in Health and Rheumatoid Diseases. Ann. Rheum Dis. 33: 366.

39. Johnson P M, Watkins J, Holbrow E J (1975) Antiglobulin production to altered IgG in Rheumatoid Arthritis The Lancet, 1(7907): 611-613

40. Johnson T, Valdimarsson H (1993). Is Measurement of Rheumatoid Factor Isotypes Clinically Useful?. Ann. Rheum. Diseases 52: 161-164.

41. Kalsi J, Isenberg D (1993) Rheumatoid factor: Primary or Secondary event in the pathogenesis of RA?. Int. Arch. Allergy Immunol. 102:209-215.

42. Kerlero de Rosbo N, Milo R, Lees M B, Burger D, Bernard C C A, Ben-Nun A (1993) Reactivity to myelin antigens in multiple sclerosis. Peripheral blood lymphocytes respond predominantly to myelin oligodendrocyte glycoprotein. J Clin. Invest. 92:2602-2608

43. Kerlero de Rosbo N, Ben-Nun A (1998) T-cell responses to myelin antigens in multiple sclerosis: Relevance of the predominant autoimmune reactivity to myelin oligodendrocyte glycoprotein J Autoimmun. 11: 287-299

44. Kramer A, Schuster A, Reineke U, Malin R, Volkmer-Engert R, Landgraf C and Schneider-Mergener J (1994) Combinatorial cellulose bound peptide libraries: Screening tools for the identification of peptides that bind ligands with pre-defined specificity. Comp. Meth. Enzymol. 6: 388-395

45. Li Q, Borovitskaya A E, DeSilva M G, Wasserfall C, MacLaren N K, Notkins A L, Lan M S (1997) Autoantigens in insulin-dependent diabetes mellitus: molecular cloning and characterisation of human IA-2 beta. Proc. Assoc. Am Physicians 109(4):420-439

46. Linington C, Webb M, Woodhams P (1984) A permanent rat T cell line that mediates experimental allergic neuritis in the Lewis rat in vivo. J. Neuroimmunol. 6:387-396

47. Linington C, Berger T, Perry L, Weerth S, Hinze-Selch D, Zhang Y, Lu H -C, Lassmann H, Werkele H. (1993) T cells specific for the myelin oligodendrocyte glycoprotein mediate an unusual autoimmune inflammatory response in the central nervous system. Eur. J. Immunol. 23:1364-1372

48. Lu J, Li Q, Xie H, Chen Z J, Borovitskaya A E, Maclaren N K, Notkins A L, Lan M S (1996) Identification of a second transmembrane protein tyrosine phosphatase, IA-2beta, as an autoantigen in insulin-dependent diabetes mellitus: precursor of the 37-kDa tryptic fragment. Proc. Natl. Acad. Sci. USA 93(6):2307-2311

49. Lukash A I, Pushkina N V, Tibulsky I E. ‘Autoantigenic properties of deamidated serum albumin, Immunologiya 68: 1987

50. Martin R (1997) Immunological aspects of experimental allergic encephalomyelitis and multiple sclerosis and their application for new therapeutic strategies, J. Neural Transm. 49:53-67

51. Mor A, Amiche M, Nicolas P, (1992) Enter a New Post-translational Modification: D-amino Acids in Gene-encoded Peptides. Elsevier Science Publishers (UK) December 1992 481-485

52. Munthe E and Natvig J B (1972). Complement Fixing Intracellular Complexes of IgG Rheumatoid Factor in Rheumatoid Plasma Cells. Scand. J. Immunol. 1: 217.

53. Murrey D E Jr. & Clarke S, Jnl of Biological Chemistry (1984) Vol. 259, No.17, September 10 pp 10722-10732 “Detection of a new site of methylation at isomerised L-aspartyl residues”

54. Nardella F A, Teller D C, Mannik M (1981). Studies on the Antigen Determinants in the Self-association of IgG Rheumatoid Factor. J. Exp. Med. 154: 112-125.

55. Natvig J B, Munthe E (1975). Self-associating IgG Rheumatoid Factor Represents a Major Response of Plasma Cells in Rheumatoid Inflammatory Tissue. Ann. NY Acad. Sci. 156: 88-95.

56. Nauert J B, Klauck T M, Langenberg L K, Scott J D (1997) Gravin, an autoantigen recognized by serum from myasthenia gravis patients, is a kinase scaffold protein Curr. Biol. 7(1):52-62

57. Nepom G T (1990). MHC Genes in HLA-associated Disease. Curr. Opinion in Immunology 2: 588-592.

58. Noritake D T, Colburn K K, Chan G. Weisbart R H (1990). Rheumatoid factors Specific for Active Rheumatoid Arthritis. Annals of the Rheumatic Diseases 49: 910-915.

59. Okamoto Y, Abe T, Niwa T, Mizuhashi S, Nishida M (1998) Development of a dual color enzyme-linked immunospot assay for simultaneous detection of murine T-helper type 1 and T-helper type 2 cells. Immunopharmacology 39: 107-116

60. Palmer J P, Asplin C M, Clemens P, Lyen K, Tatpati O, Raghu P K, Paquette T L (1983) Insulin antibodies in insulin-dependent diabetics before insulin treatment Science 222:1337-1339

61. Parekh R B, Dwek R A, Sutton B J, Fernandes D L, Leung A, Stanworth D, Rademacher T W, Mizuochi T, Taniguchi T, Matsuta K (1985). Association of Rheumatoid Arthritis and Primary Osteoarthritis with Changes in the Glycosylation Pattern of Total Serum IgG. Nature 316: 452-457.

62. Parekh R B, Isenberg D A, Ansell B M, Roitt I M, Dwek R A, Rademacher T W (1988). Galactosylation of IgG-associated Oligosaccharides is Reduced in Patients with Adult and Juvenile Onset Rheumatoid Arthritis and is Related to Disease Activity. Lancet 1: 966-999.

63. Pilkington C, Yeung E, Isenberg D, Lefvert A K, Rook G A W (1995) A galactosyl IgG and antibody specificity in rheumatoid arthritis, tuberculosis, systemic lupus erythematosus and myasthenia gravis. Autoimmunity. 22: 107-111.

64. Rawson A J, Hollander J L, Quismorio F P, Abelson N M (1969). Ann. N.Y. Acad. Sci. 168: 188

65. Ronnelid J, Klareskog L. (1997) A comparison between ELISPOT methods for the detection of cytokine producing cells: Greater sensitivity and specificity using ELISA plates as compared to nitrocellulose membranes. J Immunol. Meth. 200; 17-26

66. Schon M P, Detmar M, Parker C M (1997) Murine psoriasis-like disorder induced by naive CD4+ T cells. Nat. Med. 3(2):183-188

67. Sela M S, Fuchs (1964) On the role of charge and Optical Configuration in Antigencity. Sec. Chem. Immunol. Pp 43-56

68. Sela M, Zisman E, (1997) Different Roles of D-amino Acids in Immune Phenomena. Dept. of Immunol. Rehovot pp 449-456

69. Sun J, Link H, Olsson T, Xiao B G, Andersson G, Ekre H P, Linington C, Diener P. (1991) J. Immunol. 146:1490-1495

70. Svasti J and Milstein C (1972). The Complete Amino Acid Sequence of a Mouse κ Light Chain. Biochem. J. 128: 427-444.

71. Todome Y, Ohkuni H, Mizuse M, Furuya M, Fujikawa S, Tanaka S, Watanabe N, Fujii K, Zabriskie J, (1992) Detection of Antibodies against Streptococcal Peptidoglycan and the Peptide Subunit (Synthetic Tetra-D-Alanyl-Bovine Serum Albumin Complex) in Rheumatic-Diseases. Int. Arch Allergy Immunol 1992: 301-307

72. Tomana M, Schrohenloher R E, Koopman W J, Alarcon G S and Paul W A (1988). Abnormal Glycosylation of Serum IgG from Patients with Chronic Inflammatory Diseases. Arthritis and Rheumatism. 31: 333-338.

73. Tomiyama T, Asano S, Furiya Y, Shirasawa T, Endo N, and Morritt, Jnl of Biological Chemistry (1994) Vol. 289, No. 14, April 9, pp 10205-10208 “Racemization of Asp²³ Residue affects the aggeregation properties of Alzheimer Amyloid β Protein Analogues”

74. Van Noort J M, van Sechel A C, van Stipdonk M J B, Bajaramovic J J (1998) ‘The small heat shock protein αβ-crystallin as key autoantigen in multiple sclerosis’ In ‘Progress in brain research’ vol 117(eds: Van Leuwen F W, Salehi A, Giger R J, Holtmaat A J G D, Verhaagen J) Elsevier Science, Amsterdam

75. Watkins J, Turner M W, Roberts A.(1972). In: Peeters H (ed.) Protides of the Biological Fluids, 19th Colloquium, Oxford: 468.

76. Weir, D (ed.) (1996). The handbook of experimental immunology, vol 1., 5^{th} edn. Blackwell Scientific Publication, Oxford

77. Williams R C (1996) Autoimmune mechanisms involved in the pathogenesis of rheumatoid arthritis. Adv. Dent. Res. 10:47-51

78. Winchester R J (1975). Characterisation of IgG Complexes in Patients with Rheumatoid Arthritis. Ann. NY Acad. Sci. 256: 73-81.

79. Yoshikawa H, Lambert E H, Walser-Kuntz D R, Yasukawa Y, McCormick D J, Lennon V A (1997) A 17-Mer self-peptide of acetylcholine receptor binds to B cell MHC class II, activates helper T cells, and stimulates autoantibody production and electrophysiologic signs of myasthenia gravis. J. Immunol. 159(3):1570-1577

80. Zhang Y, Guerassimov A, Leroux J Y, Cartman A, Webber C, Lalic R. de Miguel E, Rosenberg G. Poole A R, 1998. Induction of arthritis in Balb C mice by cartilage link protein: involvement of distinct regions recognized by T and B lymphocytes. Am. J.Pathol. 153:1283-1291

79. Zisman E, Katz-Levy Y, Dayan M, Kirshner S L, Paas-Rozner M, Karni A, Abramsky O, Brautbar C, Fridkin M, Sela M, Mozes E. (1996) Peptide analogs to pathogenic epitopes of the human acetylcholine receptor alpha subunit as potential modulators of myasthenia gravis Proc. Natl. Acad. Sci. USA 93(9):4492-4497

1. A method of assay comprising subjecting a sample to a quantitative or qualitative determination of the presence in the sample of (a) an auto-reactive immune system component specifically recognising an epitope containing an isomerised peptide linkage and/or an optically inverted amino acid, and/or (b) an auto-antigen or a fragment thereof containing a said epitope and/or (c) a non-self antigen or fragment thereof which contains a said epitope and is capable of inducing an autoimmune response.

2. A method as claimed in claim 1, wherein said immune system component is a cellular immune system component.

3. A method as claimed in claim 2, wherein said immune system component is a T-lymphocyte.

4. A method as claimed in claim 1, wherein said immune system component is a humoral immune system component.

5. A method as claimed in claim 4, wherein said epitope comprises an amino acid sequence derived from IgG containing an isomerised peptide linkage or optically inverted amino acid.

6. A method as claimed in claim 4, wherein said immune system component is an auto-antibody directed against an epitope comprising the amino acid *Asx contained in any one of the sequences:
Trp-Leu-*Asx-Gly-Lys-Glu-Tyr
Trp-Glu-Ser-*Asx-Gly
His-Phe-Phe-Lys-*Asx-Ile-Val-Thr-Pro
Pro-Ser-*Asx-Glu-Gly-Lys-Gly-Arg
Ala-Leu-Gly-Ile-Gly-Thr-*Asx-Ser-Val-Ile
Trp-Ser-Phe-Gly-Ser-Glu-*Asx-Gly-Ser-Gly-*Asx-Ser-
Glu-Asn
Ala-Gly-Trp-Leu-*Asx-Gly-Ser-Val-Arg
Gly-Arg-Val-Arg-Val-*Asx-Ser-Ala-Tyr.
where Asx* is αD Asp or Asn, or is βL or βD, Asp formed by isomerisation/optical inversion of Asp or Asn residues in the original sequence.

7. A method as claimed in claim 4, wherein said immune system component is an auto-antibody directed against an epitope comprising the amino acid *Asx contained either of the sequences:
Met-Glu-Val-Gly-Trp-Tyr-Arg-Pro-Pro-Phe-Ser-Arg-
Val-Val-His-Leu-Tyr-Arg-*Asx-Gly-Lys- or
Val-Val-His-Phe-Phe-Lys-*Asx-Ile-Val-Thr-Pro
where *Asx is αD Asp or Asn, or is βD, or βL Asp formed by isomerisation/optical inversion of Asp or Asn residues in the original sequence.

8. A method as claimed in claim 4, wherein said immune system component is an auto-antibody directed against an epitope comprising the amino acid *Glx contained in any one of the sequences:
Pro-Ser-*Glx-Gly-Lys-Gly-Arg
Phe-Ser-Trp-Gly-Ala-*Glx-Gly-Arg or
Asp-Ala-*Glx-Gly-Thr-Leu-Ser-Lys
where *Glx is αD Glu or Gln, or is γL or γD Glu formed by isomerisation/optical inversion of Glu or Gln residues in the original sequence.

9. A method as claimed in any one claims 1 to 8, wherein detection of said immune system component or auto-antigen is indicative of an auto-immune disease.

10. A method as claimed in claim 9, wherein said disease is rheumatoid arthritis, multiple sclerosis, insulin dependent diabetes mellitus, myasthenia gravis, celiac disease, Chagas' disease, psoriasis, or Crohn's disease.

11. A method for the detection of an auto-antigen or fragment thereof comprising detecting the reactivity of said auto-antigen or fragment with an immunological binding partner specific for the presence in said auto-antigen of an isomerised peptide linkage or an optically inverted amino acid.

12. A method as claimed in claim 11, wherein said immuno-logical binding partner is specific for an epitope as defined in any one of claims 6 to 8.

13. A method as claimed in any preceding claim, providing information as to the amount of said immune system component or auto-antigen or non-self antigen or antigen fragment detected.

14. A method for locating an epitope or epitopes in an auto-antigen comprising using L-iso-aspartyl (D-aspartyl) methyl-transferase (IAMT) and a source of labelled methyl groups to introduce said labelled methyl groups at one or more isomerised peptide linkage and/or optically inverted amino acids in said auto-antigen, and determining at least one location in said auto-antigen at which said labelled methyl groups are thus introduced, establishing the amino acid sequence of said auto-antigen in a region encompassing a said location and testing a peptide of said amino acid sequence incorporating at said location said isomerised or optically inverted amino acid for immuno-reactivity with an auto-reactive immune system component.

15. A method as claimed in claim 14, wherein the auto-antibodies are associated with an autoimmune disease.

16. A method as claimed in claim 14, wherein the autoimmune disease is rheumatoid arthritis, multiple sclerosis, insulin dependent diabetes mellitus, myasthenia gravis, celiac disease, Chagas, disease, psoriasis, or Crohn's disease.

17. A peptide containing an epitope recognised by an auto-reactive immune system component, which epitope contains an isomerised peptide linkage and/or an optically inverted amino acid.

18. A peptide as claimed in claim 17, containing an epitope as defined in any one of claims 6 to 8.

19. A peptide as claimed in claim 18, comprising the altered amino acid residue *Asx, or *Glx and at least 3 flanking amino acid residues in the N-terminal and/or C-terminal direction, where *Glx is αD Glu or Gln, or is γL or γD Glu formed by isomerisation/optical inversion of Glu or Gln residues in the original sequence and where *Asx is αD Asp or Asn, or is βL or βD Asp formed by isomerisation/optical inversion of Asn or Asp residues in the original sequence.
